(19) United States
(12) Patent Application Publication
(10) Pub. No.: US 2023/0248231 A1
(43) Pub. Date: Aug. 10, 2023

(54) MEDICAL SYSTEM, INFORMATION PROCESSING APPARATUS, AND INFORMATION PROCESSING METHOD

(71) Applicant: SONY GROUP CORPORATION, TOKYO (JP)

(72) Inventor: KENTARO FUKAZAWA, TOKYO (JP)

(21) Appl. No.: 18/003,325

(22) PCT Filed: Jun. 2, 2021
(86) PCT No.: PCT/JP2021/020922

§ 371 Date: Dec. 26, 2022

(30) Foreign Application Priority Data
Jul. 2, 2020 (JP) 2020-114542

(51) Int. Cl. A61B 1/045; A61B 1/06; G06T 7/62; H04N 5/14; G06V 10/143; G06V 10/56; G06V 10/74

(52) U.S. Cl. CPC A61B 1/045; A61B 1/06; G06T 7/62; H04N 5/145; G06V 10/143; G06V 10/56; G06V 10/761; G06V 2201/07; G06T 2207/10024; G06T 2207/10056; G06T 2207/10068

(57) ABSTRACT

A medical system includes a light source configured to irradiate an imaging target with light having different wavelength zones in a first observation mode and a second observation mode, the imaging target being a part of a living body being operated, an imaging apparatus configured to capture reflected light from the imaging target irradiated with the light and output a captured image, a storage controller configured to perform control for causing a storage section to store a first captured image upon the first observation mode as a reference image, a generator configured to compare a second captured image upon the second observation mode with the reference image to generate a parameter used for approximating a color shade of the second captured image to a color shade of the reference image, a color conversion processor configured to perform color conversion processing on the second captured image on a basis of the parameter to output a color-converted image, and a display controller configured to perform control for causing a display section to display the color-converted image.

### FIELD

The present disclosure relates to a medical system, an information processing apparatus, and an information processing method.

### BACKGROUND

An observation of a surgical location using an image obtained by capturing a living body during an operation in the medical field can be performed in multiple types of observation modes, for example, a white light observation mode and a visible fluorescence observation mode.

### CITATION LIST

### Patent Literature

Patent Literature 1: JP 2005-348902 A

### SUMMARY

### Technical Problem

However, the technology in the related art has a limited wavelength available for color reproduction of non-fluorescent portions in the captured image, for example, in the visible fluorescence observation mode, deteriorating its color reproducibility. In other words, the captured images in the white light observation mode and the visible fluorescence observation mode will differ in color shade, which has caused a challenging issue.

Thus, the present disclosure provides a medical system, information processing apparatus, and information processing method capable of approximating the color shade of a captured image in one observation mode to the color shade of a captured image in another observation mode.

### Solution to Problem

In order to solve the above problem, a medical system according to one aspect of the present disclosure includes: a light source configured to irradiate an imaging target with light having different wavelength zones in a first observation mode and a second observation mode, the imaging target being a part of a living body being operated; an imaging apparatus configured to capture reflected light from the imaging target irradiated with the light and output a captured image; a storage controller configured to perform control for causing a storage section to store a first captured image upon the first observation mode as a reference image; a generator configured to compare a second captured image upon the second observation mode with the reference image to generate a parameter used for approximating a color shade of the second captured image to a color shade of the reference image; a color conversion processor configured to perform color conversion processing on the second captured image on a basis of the parameter to output a color-converted image; and a display controller configured to perform control for causing a display section to display the color-converted image.

### BRIEF DESCRIPTION OF DRAWINGS

FIG. 1 is a diagram illustrated to describe the background art.

FIG. 2 is a diagram illustrated to describe the background art.

FIG. 3 is a diagram illustrated to describe the overview of a first embodiment of the present disclosure.

FIG. 4 is a diagram illustrating a configuration of a medical system according to the first embodiment of the present disclosure.

FIG. 5 is a diagram illustrating a configuration of an information processing apparatus according to the first embodiment of the present disclosure.

FIG. 6 is a diagram illustrating a first configuration example of a color conversion parameter generator according to the first embodiment of the present disclosure.

FIG. 7 is a diagram illustrating a second configuration example of the color conversion parameter generator according to the first embodiment of the present disclosure.

FIG. 8 is a diagram illustrating a third configuration example of the color conversion parameter generator according to the first embodiment of the present disclosure.

FIG. 9 is a diagram illustrated to describe processing units of color conversion in a captured image in the first embodiment of the present disclosure.

FIG. 10 is a diagram illustrated to describe a color conversion parameter in a matrix format according to the first embodiment of the present disclosure.

FIG. 11 is a diagram illustrated to describe processing for preventing discontinuity in a color shade at a boundary between predetermined regions from occurring in a case of generating a color conversion parameter in units of a predetermined region in the first embodiment of the present disclosure.

FIG. 12 is a first flowchart illustrating processing performed by the information processing apparatus according to the first embodiment of the present disclosure.

FIG. 13 is a second flowchart illustrating processing performed by the information processing apparatus according to the first embodiment of the present disclosure.

FIG. 14 is a diagram illustrating a configuration of an information processing apparatus according to a second embodiment of the present disclosure.

FIG. 15 is a diagram illustrated to describe a color conversion parameter in a matrix format according to the second embodiment of the present disclosure.

FIG. 16 is a view illustrating an example of a schematic configuration of an endoscopic surgery system according to a first application example of the present disclosure.

FIG. 17 is a block diagram illustrating an example of a functional configuration of a camera head and a CCU illustrated in FIG. 16.

FIG. 18 is a view illustrating an example of a schematic configuration of a microscopic surgery system according to a second application example of the present disclosure.

FIG. 19 is a view illustrating a state of surgery in which the microscopic surgery system illustrated in FIG. 18 is used.

### DESCRIPTION OF EMBODIMENTS

The description is now given of embodiments of the present disclosure in detail with reference to the drawings. Moreover, in embodiments described below, the same components are denoted by the same reference numerals, and so a description thereof is omitted as appropriate.

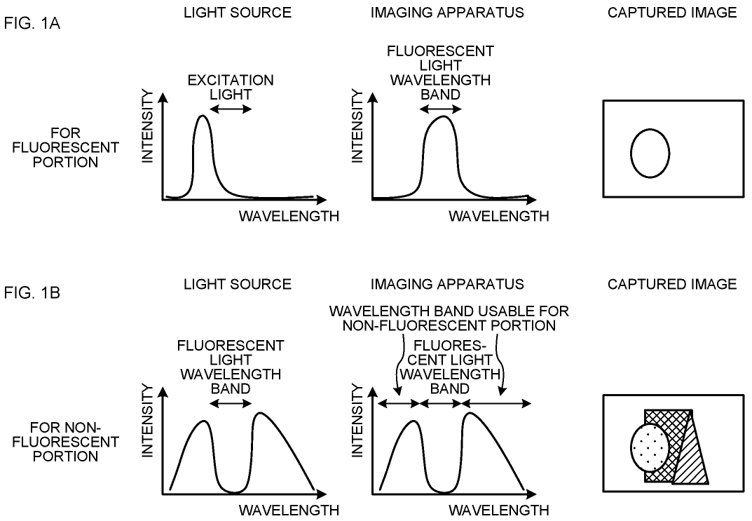

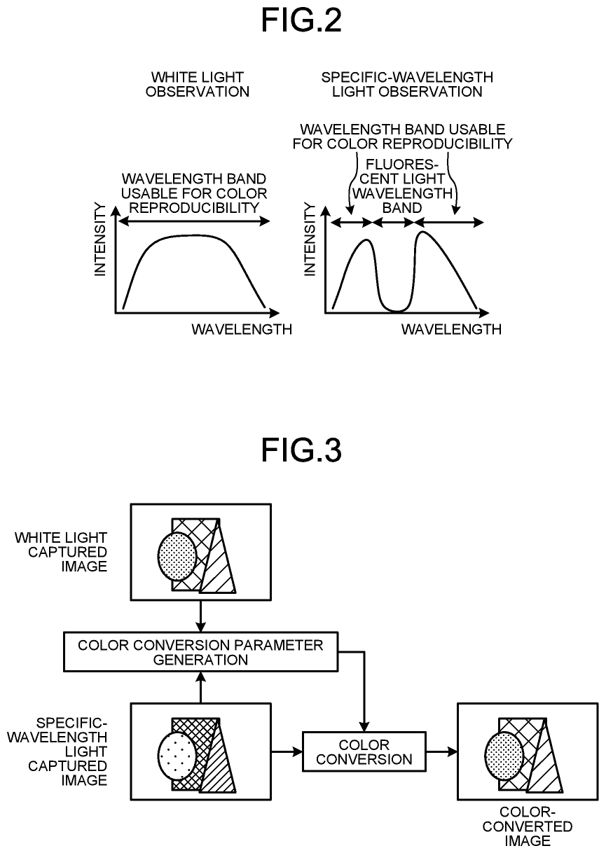

The background art is described again to facilitate understanding of embodiments. FIGS. 1 and 2 are diagrams illustrated to describe the background art. The description herein gives a visible fluorescence observation mode as an example of a specific-wavelength light observation mode. The upper part of FIG. 1 illustrates, for a fluorescent portion in the visible fluorescence observation mode, the relationship between wavelength and intensity of a light source, the relationship between wavelength and intensity used for an imaging apparatus, and the schematic view of a captured image.

Further, the lower part of FIG. 1 illustrates, for a non-fluorescent portion in the visible fluorescence observation mode, the relationship between wavelength and intensity of the light source, the relationship between wavelength and intensity used for the imaging apparatus, and the schematic view of the captured image. In other words, in this case, the wavelength zone that can be used for color reproduction of the non-fluorescent portion in an imaging apparatus is narrower than in the case of the white light observation mode, as illustrated in FIG. 2. This shows that the color reproducibility of the captured image in the visible fluorescence observation mode is degraded.

Specifically, the technology in the related art has a challenge in that the captured image in the white light observation mode can differ in color shades from the captured image in the visible fluorescence observation mode. Such an image is likely to make it difficult, for example, for the surgeon who performs a surgical operation while viewing the image to recognize the condition of the surgical location. In addition, the generation of a color conversion parameter for various photographic subjects and colors to improve color reproducibility prevents, in some cases, the relationship between input values and output results from being one-to-one with color, resulting in unsatisfactory precision. Thus, the description below gives an approach capable of approximating the color shade of a captured image in one observation mode to the color shade of a captured image in another observation mode with high precision. The description now mainly gives a case of approximating the color shade of a captured image in the visible fluorescence observation mode (an exemplary specific-wavelength light observation mode) to the color shade of a captured image in the white light observation mode.

### First Embodiment

A first embodiment is now described. FIG. 3 is a diagram illustrated to describe the overview of the first embodiment of the present disclosure. The overview of the first embodiment is as follows. A white light captured image (a captured image in the white light observation mode) is first stored. Then, a color conversion parameter (hereinafter sometimes simply referred to as “parameter”) is generated on the basis of a specific-wavelength light captured image (a captured image in the specific-wavelength light observation mode) and the white light captured image. Then, using the color conversion parameter and performing color conversion processing on the specific-wavelength light captured image to obtain a color-converted image. The color-converted image is displayed. This configuration enables using only the location (color) that appears during surgery to generate the color conversion parameter with higher precision in real-time, improving color reproducibility. The first embodiment is described in detail below.

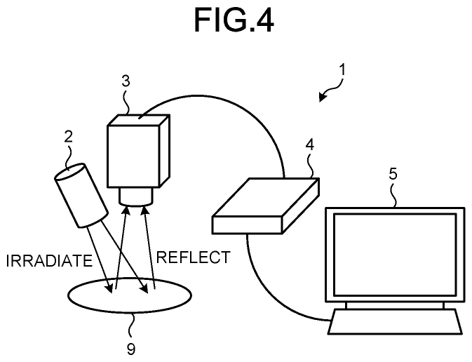

FIG. 4 is a diagram illustrating a configuration of a medical system 1 according to a first embodiment of the present disclosure. The medical system 1 according to the first embodiment roughly includes a light source 2 (light source), an imaging apparatus 3 (imaging apparatus), an information processing apparatus 4, and a display apparatus 5 (display section). The configuration of each component is described in detail below.

### (1) Light Source

The light source 2 irradiates an imaging target, which is a part of a living body being operated, with light having different wavelength zones in the white light observation mode (a first observation mode) and the visible fluorescence observation mode (a second observation mode). Moreover, although the single light source 2 is provided for simplicity of illustration in FIG. 4, such light sources can be separately provided for the white light observation mode and the visible fluorescence observation mode.

### (2) Imaging Target

An imaging target 9 (hereinafter simply referred to as “imaging target”) is a living body being operated. In one example, using the medical system 1 according to the present disclosure for microsurgery, endoscopic surgery, or the like makes it possible to perform a surgical operation while identifying the positions of internal organs, blood vessels, and the like. Thus, it is possible to achieve a safer and more precise surgical operation, contributing to further development of medical technology.

### (3) Imaging Apparatus

The imaging apparatus 3 captures reflected light from the imaging target irradiated with light and outputs a captured image. The imaging apparatus 3 is, for example, an imager. Moreover, although the single imaging apparatus 3 is provided for simplicity of illustration in FIG. 4, such imaging apparatuses can be separately provided for the white light observation mode and the visible fluorescence observation mode.

### (4) Information Processing Apparatus

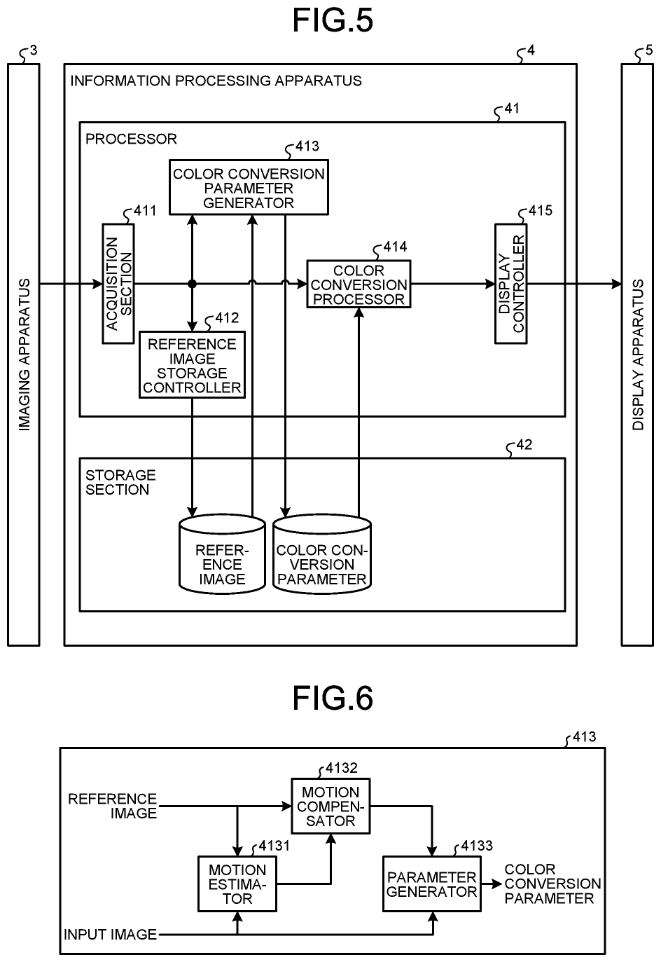

The description is now given of the information processing apparatus 4 with reference to FIG. 5. FIG. 5 is a diagram illustrating a functional configuration example of the information processing apparatus 4 according to the first embodiment of the present disclosure. The information processing apparatus 4 is an image processing device and includes a processor 41 and a storage section 42 as main components.

The processor 41 is implemented as, for example, a central processing unit (CPU) and includes an acquisition section 411, a reference image storage controller 412 (or a storage controller), a color conversion parameter generator 413 (or a generator), a color conversion processor 414, and a display controller 415.

The acquisition section 411 acquires a white light captured image upon the white light observation mode and a visible fluorescence captured image in the visible fluorescence observation mode from the imaging apparatus 3.

The reference image storage controller 412 performs control for causing the storage section 42 to store the white light captured image upon the white light observation mode as a reference image. The reference image storage controller 412 causes the storage section 42 to store, for example, the white light captured image upon the white light observation mode as a reference image by default.

Further, the reference image storage controller 412 causes the storage section 42 to store the white light captured image upon the white light observation mode as a reference image in the case where, for example, the area of an object (such as a surgical tool) other than a living body in the image is less than or equal to a predetermined proportion. In addition, in this case, suppose the reference image storage controller 412, which has caused the storage section 42 to store the white light captured image as a reference image, acquires a white light captured image having a smaller area of an object other than a living body. In this case, the reference image storage controller 412 can update the reference image to the acquired white light captured image.

Further, the reference image storage controller 412 causes the storage section 42 to store the white light captured image upon the white light observation mode as a reference image in the case where, for example, the definition of the image is greater than or equal to a predetermined threshold. It is conceivable that the definition of an image less than the predetermined threshold is a cause, for example, that the imaging apparatus 3 has moved. In addition, in that case, suppose the reference image storage controller 412, which has caused the storage section 42 to store the white light captured image as a reference image, acquires a white light captured image with a higher definition. In this case, the reference image storage controller 412 can update the reference image to the acquired white light captured image.

Further, the reference image storage controller 412 causes the storage section 42 to store the white light captured image upon the white light observation mode as a reference image in the case where, for example, the size and position of a surgical target part (e.g., such as an internal organ) satisfy a predetermined condition. This configuration makes it possible to reduce the possibility that there is a location that appears in the reference image or the visible fluorescence captured image but not in the other, increasing the precision of the color conversion parameter. In addition, in this case, suppose the reference image storage controller 412, which has caused the storage section 42 to store the white light captured image as a reference image, acquires a white light captured image having a more suitable size and position of the surgical target part. In this case, the reference image storage controller 412 can update the reference image to the acquired white light captured image.

Further, the reference image storage controller 412 causes the storage section 42 to store the white light captured image upon the white light observation mode as a reference image at a timing specified by a user. This configuration makes it easier to reproduce the target color intended by the user (the color shade of the reference image) in the visible fluorescence captured image.

Further, the reference image storage controller 412 causes the storage section 42 to store a plurality of white light captured images upon the white light observation mode as reference images. In this case, the plurality of white light captured images stored in the storage section 42 as reference images can be obtained, for example, by default at any optional time intervals. This configuration makes it possible to store a plurality of reference images with simple processing.

Further, the plurality of white light captured images stored in the storage section 42 as reference images can have, for example, different image characteristics. This configuration makes it possible to reduce the possibility of decreasing the precision of color conversion due to the use of a reference image with special image characteristics.

Further, the plurality of white light captured images stored in the storage section 42 as reference images can be acquired, for example, at a timing specified by a user. This makes it possible to store a plurality of reference images by a user considering various conditions.

The color conversion parameter generator 413 compares the visible fluorescence captured image upon the visible fluorescence observation mode with the reference image to generate a parameter used to approximate the color shade of the visible fluorescence captured image to the color shade of the reference image. In this case, the color conversion parameter generator 413 generates the parameter in units of a pixel, in units of a predetermined region that includes multiple pixels, or in units of the whole image.

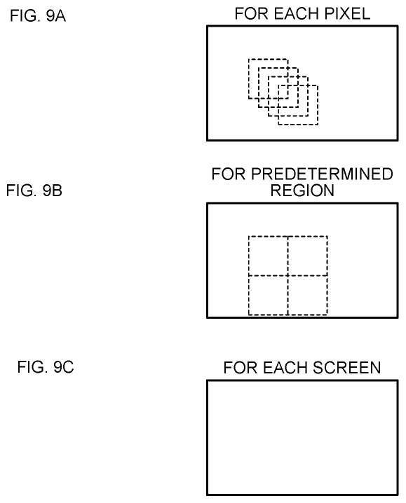

In this regard, FIG. 9 is a diagram illustrated to describe the units of processing of color conversion processing in a captured image in the first embodiment of the present disclosure. The color conversion processing in a captured image can be performed, for example, for each pixel as illustrated in portion (a), for each predetermined region as illustrated in portion (b), or for each screen (whole image) as illustrated in portion (c).

In the case of the portion (a), the color conversion parameter is generated for each pixel. In this case, information with any optional range centered on a noticed pixel can be used. Generating the parameter for each pixel can have higher precision than in the case of generating the parameter for each screen.

In the case of portion (b), the color conversion parameter is generated for each predetermined region. Generating the parameter for each predetermined region can have higher precision than in the case of generating the parameter for each screen.

In the case of portion (c), the color conversion parameter is generated for each screen. Generating the parameter for each screen makes it possible to make the color conversion parameter with simple processing.

Referring back to FIG. 5, in the case of generating a parameter in units of pixels or units of predetermined regions, the color conversion parameter generator 413 generates the parameter by performing motion estimation and motion compensation of a subject to align positions of the subject. In addition, the color conversion parameter generator 413 performs, for example, the identification of internal organs in the visible fluorescence captured image and generates the parameter for each internal organ.

Then, FIG. 6 is a diagram illustrating a first configuration example of the color conversion parameter generator 413 according to the first embodiment of the present disclosure. A motion estimator 4131 uses the reference image and an input image (visible fluorescence reference image) to estimate the motion of a subject on the basis of the feature value in each image. In addition, a motion compensator 4132 performs motion compensation on it on the basis of the reference image and a result obtained by the estimation in the motion estimator 4131. Then, a parameter generator 4133 generates the color conversion parameter on the basis of the input image and a result obtained by the motion compensation in the motion compensator 4132.

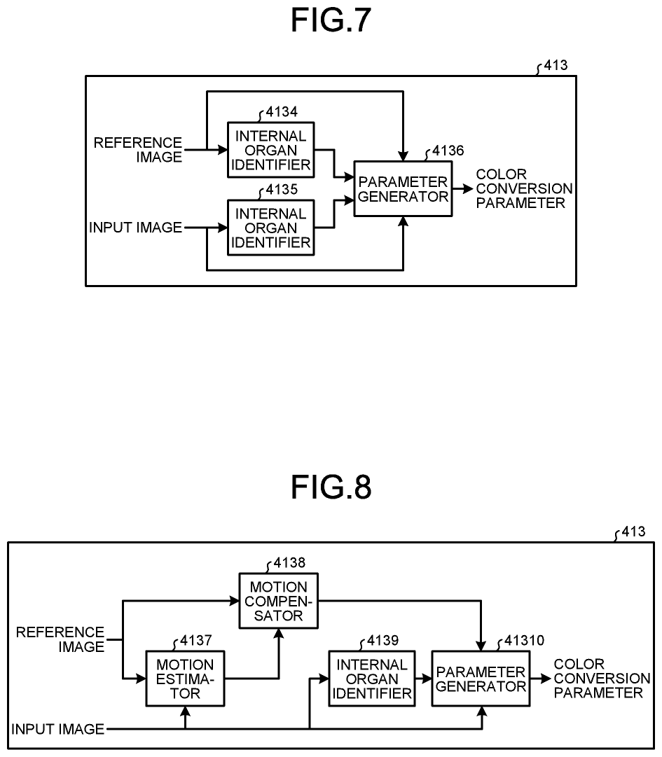

Further, FIG. 7 is a diagram illustrating a second configuration example of the color conversion parameter generator 413 according to the first embodiment of the present disclosure. An internal organ identifier 4134 identifies an internal organ on the reference image. In addition, an internal organ identifier 4135 identifies an internal organ in the input image. Then, a parameter generator 4136 generates a color conversion parameter on the basis of the reference image, the input image, a result obtained by identifying the internal organ in the internal organ identifier 4134, and a result obtained by identifying the internal organ in the internal organ identifier 4135.

FIG. 8 is a diagram illustrating a third configuration example of the color conversion parameter generator 413 according to the first embodiment of the present disclosure. A motion estimator 4137 estimates the motion of a subject on the basis of the feature value in each image using the reference image and the input image. In addition, a motion compensator 4138 compensates for the motion on the basis of a result obtained by the estimation in the motion estimator 4137 and the reference image. In addition, an internal organ identifier 4139 identifies an internal organ on the input image. Then, a parameter generator 41310 generates color conversion parameters on the basis of the input image, a result obtained by identifying the internal organ in the internal organ identifier 4139, and a result obtained by compensating the motion in the motion compensator 4138.

Referring back to FIG. 5, the color conversion parameter generator 413 generates the parameter so that discontinuity in the color shade at a boundary between pixels or between predetermined regions can be prevented from occurring, for example, in the case of generating the parameter in units of pixels or units of predetermined regions. In this regard, FIG. 11 is a diagram illustrated to describe processing for preventing discontinuity in the color shade at a boundary between predetermined regions from occurring in the case of generating the color conversion parameter for each predetermined region in the first embodiment of the present disclosure.

The color conversion parameter generator 413 performs, for example, interpolation processing (e.g., linear interpolation processing) so that discontinuity in the color shade at a boundary between predetermined regions can be prevented from occurring after generating a parameter for each predetermined region. In the example of FIG. 11, a result obtained by interpolating color conversion parameters of four regions surrounded by the broken line can be used, for example, as a color conversion parameter for a pixel A.

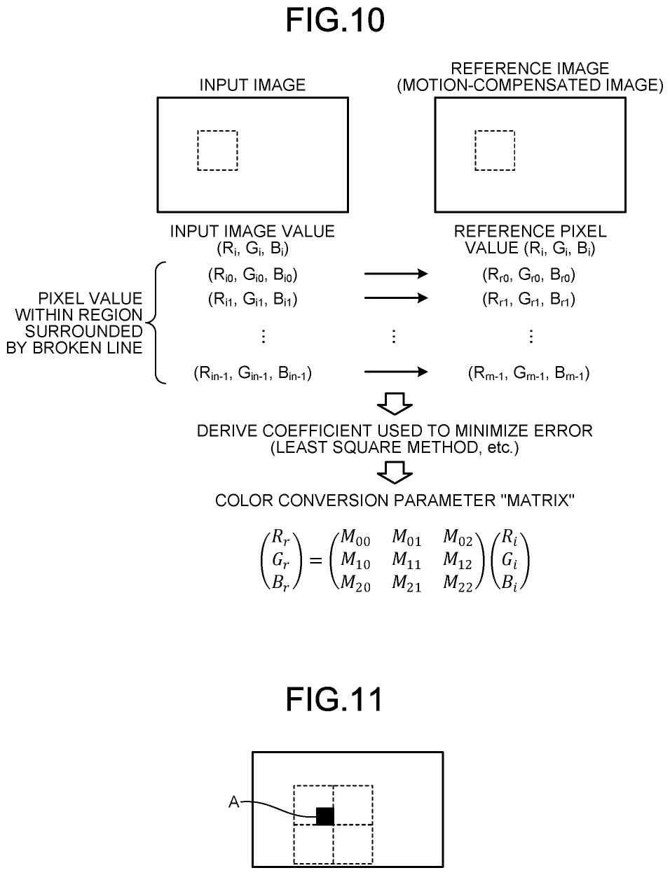

Referring back to FIG. 5, the color conversion parameter generator 413 generates, for example, a parameter in a matrix format used to minimize a difference in color shades between the image subjected to color conversion that is obtained by subjecting the visible fluorescence captured image to the color conversion and the reference image. In this regard, FIG. 10 is a diagram illustrated to describe the color conversion parameter in a matrix format according to the first embodiment of the present disclosure. The units for generating the color conversion parameter can be a pixel, a predetermined region, a screen, or an internal organ. In addition, in the case of using a plurality of reference images, any optional number of reference images among them can be used. Still, the motion estimation and compensation on a subject are performed as necessary. In this case, weighting can be subjected depending on the reliability of motion estimation and motion compensation.

As illustrated in FIG. 10, the color conversion parameter generator 413 uses an input pixel value of the input image and a reference pixel value of the reference image (motion-compensated image) to derive a coefficient used to minimize the error (difference) using the least square method, generating the color conversion parameter in a matrix format. Moreover, although the RGB (red, green, and blue) color space is used as the color space herein, any optional color space can be employed. In this way, using the parameter in matrix format makes it possible to perform the color conversion with simple processing.

Referring back to FIG. 5, the color conversion parameter generator 413 generates, for example, a parameter in a lookup table format used to minimize a difference in color shade between the image subjected to color conversion obtained by subjecting the visible fluorescence captured image to the color conversion and the reference image. This configuration makes it possible to perform non-linear processing, achieving high-precision color reproduction.

Further, the color conversion parameter generator 413 generates the parameter using, for example, machine learning. This configuration makes it possible to use, for example, machine learning using a predetermined amount or more of teacher data, achieving high-precision color reproduction.

Further, the timing of generating the parameter can be set, for example, immediately after switching from the white light observation mode into the visible fluorescence observation mode. Alternatively, the timing can be set after several frames from the switching. In addition, the timing can be set every frame or at any frame intervals. In addition, the parameter can be subjected to smoothing in the time direction. Alternatively, the timing can be set by a user. In this way, the timing of generating the parameter can be determined by the user considering the simplicity of processing (calculation cost), color reproducibility, and the like.

The color conversion processor 414 performs the color conversion processing on the visible fluorescence captured image on the basis of the parameter generated by the color conversion parameter generator 413 and outputs a color-converted image in the case of the visible fluorescence observation mode targeted for color conversion processing.

The display controller 415 performs various control functions for display representations. The display controller 415 performs control for causing, for example, the display apparatus 5 to display the color-converted image.

The storage section 42 stores various types of information. The storage section 42 stores, for example, the reference image, color conversion parameter, a result obtained by the calculation in each component of the processor 41, and the like. Moreover, a storage apparatus external to the medical system 1 can be employed instead of the storage section 42.

### (5) Display Apparatus

The display apparatus 5 displays various types of information under the control of the display controller 415. The display apparatus 5 displays, for example, the color-converted image output by the color conversion processor 414. Moreover, a display apparatus external to the medical system 1 can be employed instead of the display apparatus 5.

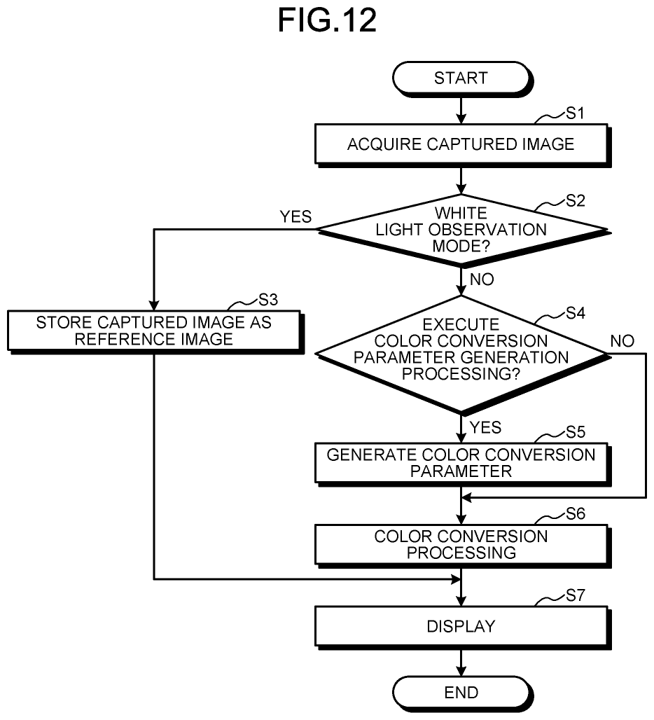

FIG. 12 is a first flowchart illustrating processing by the information processing apparatus 4 according to the first embodiment of the present disclosure. In step S1, the acquisition section 411 acquires a captured image from the imaging apparatus 3.

Next, in step S2, the reference image storage controller 412 determines whether or not the mode is the white light observation mode; then, if the result is Yes, proceed to step S3, but if No, proceed to step S4. In step S3, the reference image storage controller 412 causes the storage section 42 to store the captured image as a reference image. Moreover, as described above, it is also possible for the reference image storage controller 412 to perform the processing of step S3 only if a predetermined condition is satisfied (e.g., the area of an object other than the living body in an image is equal to or less than a predetermined proportion).

In step S4, the color conversion parameter generator 413 determines whether or not to execute the color conversion parameter generation processing; then, if the result is Yes, proceed to step S5, but if No, proceed to step S6.

In step S5, the color conversion parameter generator 413 compares the visible fluorescence captured image with the reference image to generate a parameter used for approximating the color shade of the visible fluorescence captured image to the color shade of the reference image.

After step S5 or if the result is No in step S4, in step S6, the color conversion processor 414 performs the color conversion processing on the visible fluorescence captured image on the basis of the parameter generated in step S5 and outputs the color-converted image.

After step S6, in step S7, the display controller 415 performs control for causing the display apparatus 5 to display the color-converted image that is output in step S6.

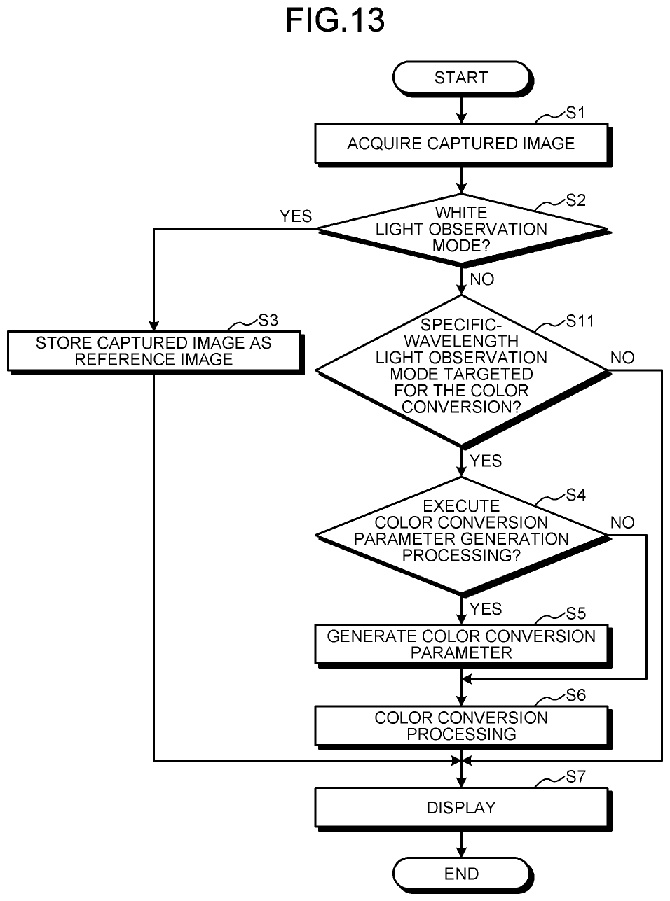

Moreover, in the processing of FIG. 12, the visible fluorescence captured image is subjected to color conversion by default upon the visible fluorescence observation mode. However, in the case where there are multiple specific-wavelength light observation modes and the specific-wavelength light observation mode targeted for the color conversion is specified, it is possible to perform the processing as illustrated in FIG. 13.

FIG. 13 is a second flowchart illustrating processing performed by the information processing apparatus 4 according to the first embodiment of the present disclosure. Steps S1 and S2 are similar to those in FIG. 12. After step S2, in step S3, the color conversion parameter generator 413 determines whether or not the mode is the specific-wavelength light observation mode targeted for the color conversion. If the result is Yes, proceed to step S4, but if No, proceed to step S7. Steps S3 to S7 are similar to those in FIG. 12.

As described above, the information processing apparatus 4 of the first embodiment stores the white light captured image as the reference image and then, upon acquiring the visible fluorescence captured image, compares the white light captured image with the reference image, resulting in generating the color conversion parameter. The information processing apparatus 4 performs the color conversion processing on the visible fluorescence captured image on the basis of the generated color conversion parameter. Such configuration makes it possible to approximate the color shade of the visible fluorescence captured image to the color shade of the white light captured image with simple processing and in real-time.

Thus, visibility similar to that in the white light observation mode is maintained even in the visible fluorescence observation mode, which facilitates the identification of, for example, a fluorescent portion and its surrounding non-fluorescent portion. This improves the safety of surgery. In addition, this configuration eliminates the need for switching between the white light observation mode and the visible fluorescence observation mode halfway, which is convenient.

In one example, the technology in related art stores a color correction coefficient for each internal organ in advance, identifies the internal organ, and corrects the color for the identified internal organ. However, this method depends on the usage environment, leading to the difference in the internal organ colors between when previously calculating the color correction coefficient and when using it, which may fail to convert the color into an appropriate color. In one example, the possible causes include the type of light source, changes in the performance of the light source over time, the type of lens (hard lens), and differences in the color of internal organs from person to person. The information processing apparatus 4 according to the present disclosure generates the color conversion parameter in real-time, preventing it from being unaffected by the usage environment.

### Second Embodiment

A second embodiment is now described. Descriptions of parts similar to those of the first embodiment are omitted as appropriate. The second embodiment differs from the first embodiment in that it refers to the color-converted image in advance and weights it to generate the color conversion parameter.

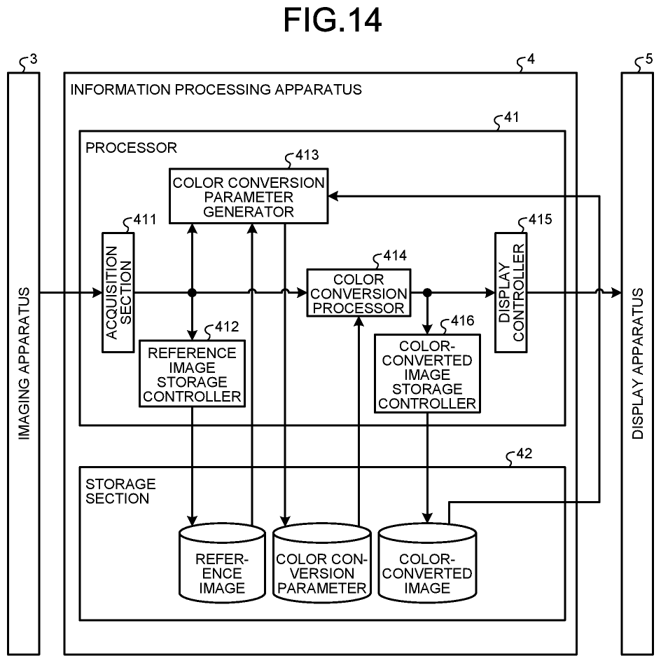

FIG. 14 is a diagram illustrating the configuration of an information processing apparatus 4 according to the second embodiment of the present disclosure. In the processor 41 of the information processing apparatus 4, a color-converted image storage controller 416 is additionally provided. The color-converted image storage controller 416 performs control for causing the storage section 42 to store the color-converted image output by the color conversion processor 414. Then, the color conversion parameter generator 413 generates the parameter further on the basis of the color-converted image stored in the storage section 42.

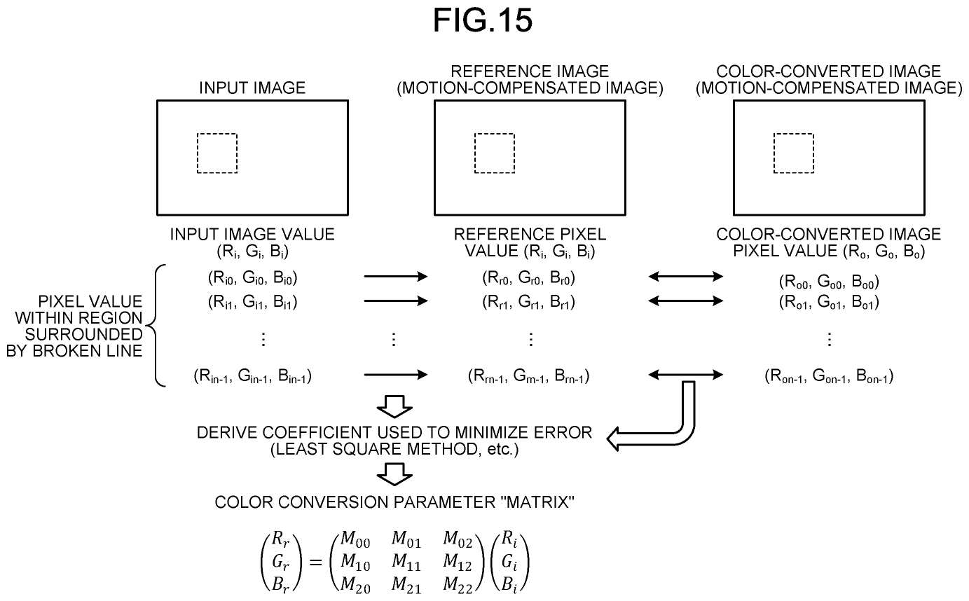

FIG. 15 is a diagram illustrated to describe a color conversion parameter in a matrix format according to the second embodiment of the present disclosure. Descriptions of parts similar to those in FIG. 10 are omitted as appropriate. As illustrated in FIG. 15, the color conversion parameter generator 413 uses an input pixel value of the input image and a reference pixel value of the reference image (motion-compensated image) to derive a coefficient used to minimize the error (difference) using the least square method or the like, generating the color conversion parameter in a matrix format. In this case, the color conversion parameter generator 413 weights the difference (error) between pixel values for each pixel on the basis of the color-converted image (motion-compensated image).

As described above, the information processing apparatus 4 according to the second embodiment achieves the effect capable of reducing or preventing the difference in color reproducibility due to the difference in color, in addition to the effects of the first embodiment.

### Third Embodiment

A third embodiment is now described. Descriptions of parts similar to those of the first embodiment are omitted as appropriate. The third embodiment differs from the first embodiment in that it refers to a result obtained by specifying a color reproduction priority location (color) by the user and weights it to generate the color conversion parameter.

The color conversion parameter generator 413 generates the parameter so that the color shade of the visible fluorescence captured image for the location (color) specified by the user in the living body approximates the color shade of the reference image. This location to be specified by the user can be implemented using, for example, a predetermined user interface (UI).

As described above, the information processing apparatus 4 according to the third embodiment achieves the effect capable of improving the color reproducibility of the location (color) that the user places priority on, in addition to the effects of the first embodiment.

### First Application Example

The technology according to the present disclosure is applicable to various products. In one example, the technology according to the present disclosure is applicable to an endoscopic system. Hereinafter, an endoscopic surgery system as an example of an endoscope surgery system will be described.

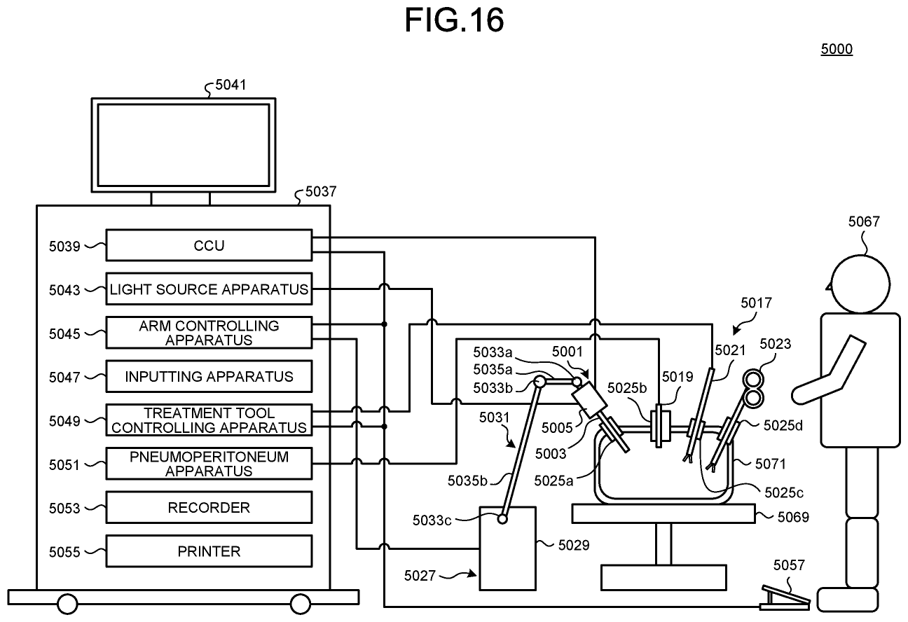

FIG. 16 is a view illustrating an example of a schematic configuration of an endoscopic surgery system 5000 to which the technology according to the present disclosure can be applied. In FIG. 16, a state is illustrated in which a surgeon (medical doctor) 5067 is using the endoscopic surgery system 5000 to perform surgery for a patient 5071 on a patient bed 5069. As illustrated, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d is used to puncture the abdominal wall. Then, a lens barrel 5003 of the endoscope 5001 and the other surgical tools 5017 are inserted into body cavity of the patient 5071 through the trocars 5025a to 5025d. In the example illustrated, as the other surgical tools 5017, a pneumoperitoneum tube 5019, an energy device 5021 and forceps 5023 are inserted into body cavity of the patient 5071. Further, the energy device 5021 is a treatment tool for performing incision and peeling of a tissue, sealing of a blood vessel or the like by high frequency current or ultrasonic vibration. However, the surgical tools 5017 illustrated are mere examples at all, and as the surgical tools 5017, various surgical tools which are generally used in endoscopic surgery such as, for example, tweezers or a retractor may be used.

An image of a surgical location in a body cavity of the patient 5071 imaged by the endoscope 5001 is displayed on a display apparatus 5041. The surgeon 5067 would use the energy device 5021 or the forceps 5023 while watching the image of the surgical location displayed on the display apparatus 5041 on the real time basis to perform such treatment as, for example, resection of an affected area. It is to be noted that, though not illustrated, the pneumoperitoneum tube 5019, the energy device 5021 and the forceps 5023 are supported by the surgeon 5067, an assistant or the like during surgery.

### Supporting Arm Apparatus

The supporting arm apparatus 5027 includes an arm unit 5031 extending from a base unit 5029. In the example illustrated, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endoscope 5001 can be implemented.

### Endoscope

The endoscope 5001 includes the lens barrel 5003 which has a region of a predetermined length from a distal end thereof to be inserted into a body cavity of the patient 5071, and a camera head 5005 connected to a proximal end of the lens barrel 5003. In the example illustrated, the endoscope 5001 is illustrated as a rigid endoscope having the lens barrel 5003 of the hard type. However, the endoscope 5001 may otherwise be configured as a flexible endoscope having the lens barrel 5003 of the flexible type.

The lens barrel 5003 has, at the distal end thereof, an opening in which an objective lens is fitted. A light source apparatus 5043 is connected to the endoscope 5001 such that light generated by the light source apparatus 5043 is introduced to the distal end of the lens barrel by a light guide extending in the inside of the lens barrel 5003 and is irradiated toward an observation target in a body cavity of the patient 5071 through the objective lens. It is to be noted that the endoscope 5001 may be a forward-viewing endoscope or may be an oblique-viewing endoscope or a side-viewing endoscope.

An optical system and an image sensor are provided in the inside of the camera head 5005 such that reflected light (observation light) from an observation target is condensed on the image sensor by the optical system. The observation light is photoelectrically converted by the image sensor to generate an electric signal corresponding to the observation light, namely, an image signal corresponding to an observation image. The image signal is transmitted as RAW data to a camera control unit (CCU) 5039. It is to be noted that the camera head 5005 has a function incorporated therein for suitably driving the optical system of the camera head 5005 to adjust the magnification and the focal distance.

It is to be noted that, in order to establish compatibility with, for example, a stereoscopic vision (three dimensional (3D) display), a plurality of image sensors may be provided on the camera head 5005. In this case, a plurality of relay optical systems is provided in the inside of the lens barrel 5003 in order to guide observation light to each of the plurality of image sensors.

### Various Apparatus Incorporated in Cart

The CCU 5039 includes a central processing unit (CPU), a graphics processing unit (GPU) or the like and integrally controls operation of the endoscope 5001 and the display apparatus 5041. In particular, the CCU 5039 performs, for an image signal received from the camera head 5005, various image processes for displaying an image based on the image signal such as, for example, a development process (demosaic process), The CCU 5039 provides the image signal for which the image processes have been performed to the display apparatus 5041. Further, the CCU 5039 transmits a control signal to the camera head 5005 to control driving of the camera head 5005. The control signal may include information relating to an image capturing condition such as a magnification or a focal distance.

The display apparatus 5041 displays an image based on an image signal for which the image processes have been performed by the CCU 5039 under the control of the CCU 5039. If the endoscope 5001 is ready for imaging of a high resolution such as 4K (horizontal pixel number 3840×vertical pixel number 2160), 8K (horizontal pixel number 7680×vertical pixel number 4320) or the like and/or ready for 3D display, then a display apparatus by which corresponding display of the high resolution and/or 3D display are possible may be used as the display apparatus 5041. Where the apparatus is ready for imaging of a high resolution such as 4K or 8K, if the display apparatus used as the display apparatus 5041 has a size of equal to or not less than 55 inches, then a more immersive experience can be obtained. Further, a plurality of display apparatus 5041 having different resolutions and/or different sizes may be provided in accordance with purposes.

The light source apparatus 5043 includes a light source such as, for example, a light emitting diode (LED) and supplies irradiation light for imaging of a surgical location to the endoscope 5001.

The arm controlling apparatus 5045 includes a processor such as, for example, a CPU and operates in accordance with a predetermined program to control driving of the arm unit 5031 of the supporting arm apparatus 5027 in accordance with a predetermined control method.

An inputting apparatus 5047 is an input interface for the endoscopic surgery system 5000. A user can perform inputting of various kinds of information or instruction inputting to the endoscopic surgery system 5000 through the inputting apparatus 5047. For example, the user would input various kinds of information relating to surgery such as physical information of a patient, information regarding a surgical procedure of the surgery and so forth through the inputting apparatus 5047. Further, the user would input, for example, an instruction to drive the arm unit 5031, an instruction to change an image capturing condition (type of irradiation light, magnification, focal distance or the like) by the endoscope 5001, an instruction to drive the energy device 5021 or the like through the inputting apparatus 5047.

The type of the inputting apparatus 5047 is not limited and may be that of any one of various known inputting apparatus. As the inputting apparatus 5047, for example, a mouse, a keyboard, a touch panel, a switch, a foot switch 5057 and/or a lever or the like may be applied. Where a touch panel is used as the inputting apparatus 5047, it may be provided on the display face of the display apparatus 5041.

Otherwise, the inputting apparatus 5047 is a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5047 includes a camera which can detect a motion of a user, and various kinds of inputting are performed in response to a gesture or a line of sight of a user detected from a video captured by the camera. Further, the inputting apparatus 5047 includes a microphone which can collect the voice of a user, and various kinds of inputting are performed by voice collected by the microphone. By configuring the inputting apparatus 5047 such that various kinds of information can be input in a contactless fashion in this manner, especially a user who belongs to a clean area (for example, the surgeon 5067) can operate an apparatus belonging to an unclean area in a contactless fashion. Further, since the user can operate an apparatus without releasing a possessed surgical tool from its hand, the convenience to the user is improved.

A treatment tool controlling apparatus 5049 controls driving of the energy device 5021 for cautery or incision of a tissue, sealing of a blood vessel or the like. A pneumoperitoneum apparatus 5051 feeds gas into a body cavity of the patient 5071 through the pneumoperitoneum tube 5019 to inflate the body cavity in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

In the following, especially a characteristic configuration of the endoscopic surgery system 5000 is described in more detail.

### Supporting Arm Apparatus

The supporting arm apparatus 5027 includes the base unit 5029 serving as a base, and the arm unit 5031 extending from the base unit 5029. In the example illustrated, the arm unit 5031 includes the plurality of joint portions 5033a, 5033b and 5033c and the plurality of links 5035a and 5035b connected to each other by the joint portion 5033b. In FIG. 16, for simplified illustration, the configuration of the arm unit 5031 is illustrated in a simplified form. Actually, the shape, number and arrangement of the joint portions 5033a to 5033c and the links 5035a and 5035b and the direction and so forth of axes of rotation of the joint portions 5033a to 5033c can be set suitably such that the arm unit 5031 has a desired degree of freedom. For example, the arm unit 5031 may preferably be configured such that it has a degree of freedom equal to or not less than 6 degrees of freedom. This makes it possible to move the endoscope 5001 freely within the movable range of the arm unit 5031. Consequently, it becomes possible to insert the lens barrel 5003 of the endoscope 5001 from a desired direction into a body cavity of the patient 5071.

An actuator is provided in each of the joint portions 5033a to 5033c, and the joint portions 5033a to 5033c are configured such that they are rotatable around predetermined axes of rotation thereof by driving of the respective actuators. The driving of the actuators is controlled by the arm controlling apparatus 5045 to control the angle of rotation of each of the joint portions 5033a to 5033c thereby to control driving of the arm unit 5031. Consequently, control of the position and the posture of the endoscope 5001 can be implemented. Thereupon, the arm controlling apparatus 5045 can control driving of the arm unit 5031 by various known control methods such as force control or position control.

For example, if the surgeon 5067 suitably performs operation inputting through the inputting apparatus 5047 (including the foot switch 5057), then driving of the arm unit 5031 may be controlled suitably by the arm controlling apparatus 5045 in response to the operation input to control the position and the posture of the endoscope 5001. After the endoscope 5001 at the distal end of the arm unit 5031 is moved from an arbitrary position to a different arbitrary position by the control just described, the endoscope 5001 can be supported fixedly at the position after the movement. It is to be noted that the arm unit 5031 may be operated in a master-slave fashion. In this case, the arm unit 5031 may be remotely controlled by the user through the inputting apparatus 5047 which is placed at a place remote from the operating room.

Further, where force control is applied, the arm controlling apparatus 5045 may perform power-assisted control to drive the actuators of the joint portions 5033a to 5033c such that the arm unit 5031 may receive external force by the user and move smoothly following the external force. This makes it possible to move, when the user directly touches the arm unit 5031 and moves the arm unit 5031, the arm unit 5031 with comparatively weak force. Accordingly, it becomes possible for the user to move the endoscope 5001 more intuitively by a simpler and easier operation, and the convenience to the user can be improved.

Here, generally in endoscopic surgery, the endoscope 5001 is supported by a medical doctor called scopist. In contrast, where the supporting arm apparatus 5027 is used, the position of the endoscope 5001 can be fixed more certainly without hands, and therefore, an image of a surgical location can be obtained stably and surgery can be performed smoothly.

It is to be noted that the arm controlling apparatus 5045 may not necessarily be provided on the cart 5037. Further, the arm controlling apparatus 5045 may not necessarily be a single apparatus. For example, the arm controlling apparatus 5045 may be provided in each of the joint portions 5033a to 5033c of the arm unit 5031 of the supporting arm apparatus 5027 such that the plurality of arm controlling apparatus 5045 cooperates with each other to implement driving control of the arm unit 5031.

### Light Source Apparatus

The light source apparatus 5043 supplies irradiation light upon imaging of a surgical location to the endoscope 5001. The light source apparatus 5043 includes a white light source which includes, for example, an LED, a laser light source or a combination of them. In this case, where a white light source includes a combination of red, green, and blue (RGB) laser light sources, since the output intensity and the output timing can be controlled with high accuracy for each color (each wavelength), adjustment of the white balance of a captured image can be performed by the light source apparatus 5043. Further, in this case, if laser beams from the respective RGB laser light sources are irradiated time-divisionally on an observation target and driving of the image sensors of the camera head 5005 is controlled in synchronism with the irradiation timings, then images individually corresponding to the R, G and B colors can be captured time-divisionally. According to the method just described, a color image can be obtained even if a color filter is not provided for the image sensor.

Further, driving of the light source apparatus 5043 may be controlled such that the intensity of light to be output is changed for each predetermined time. By controlling driving of the image sensor of the camera head 5005 in synchronism with the timing of the change of the intensity of light to acquire images time-divisionally and synthesizing the images, an image of a high dynamic range free from underexposed blocked up shadows and overexposed highlights can be created.

Further, the light source apparatus 5043 may be configured to be able to supply light of a predetermined wavelength band ready for specific-wavelength light. In specific-wavelength light observation, for example, by utilizing the wavelength dependency of absorption of light in a body tissue to irradiate with light of a narrower wavelength band in comparison with irradiation light upon ordinary observation (namely, white light), narrow band light observation (narrow band imaging) of imaging a predetermined tissue such as a blood vessel of a superficial portion of the mucous membrane or the like in a high contrast is performed. Alternatively, in specific-wavelength light observation, fluorescence observation for obtaining an image from fluorescence generated by irradiation of excitation light may be performed. In fluorescence observation, it is possible to perform observation of fluorescence from a body tissue by irradiating the body tissue with excitation light (autofluorescence observation) or to obtain a fluorescent light image by locally injecting a reagent such as indocyanine green (ICG) into a body tissue and irradiating the body tissue with excitation light corresponding to a fluorescent light wavelength of the reagent. The light source apparatus 5043 can be configured to be able to supply such narrow-band light and/or excitation light suitable for specific-wavelength light observation as described above.

### Camera Head and CCU

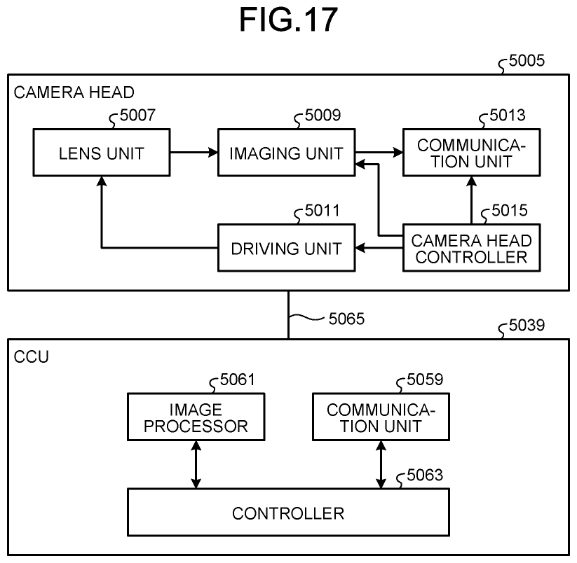

Functions of the camera head 5005 of the endoscope 5001 and the CCU 5039 are described in more detail with reference to FIG. 17. FIG. 17 is a block diagram illustrating an example of a functional configuration of the camera head 5005 and the CCU 5039 illustrated in FIG. 16.

Referring to FIG. 17, the camera head 5005 has, as functions thereof, a lens unit 5007, an imaging unit 5009, a driving unit 5011, a communication unit 5013 and a camera head controller 5015. Further, the CCU 5039 has, as functions thereof, a communication unit 5059, an image processor 5061 and a controller 5063. The camera head 5005 and the CCU 5039 are connected to be bidirectionally communicable to each other by a transmission cable 5065.

First, a functional configuration of the camera head 5005 is described. The lens unit 5007 is an optical system provided at a connecting location of the camera head 5005 to the lens barrel 5003. Observation light taken in from the distal end of the lens barrel 5003 is introduced into the camera head 5005 and enters the lens unit 5007. The lens unit 5007 includes a combination of a plurality of lenses including a zoom lens and a focusing lens. The lens unit 5007 has optical properties adjusted such that the observation light is condensed on a light receiving face of the image sensor of the imaging unit 5009. Further, the zoom lens and the focusing lens are configured such that the positions thereof on their optical axis are movable for adjustment of the magnification and the focal point of a captured image.

The imaging unit 5009 includes an image sensor, and is disposed at a succeeding stage to the lens unit 5007. Observation light having passed through the lens unit 5007 is condensed on the light receiving face of the image sensor, and an image signal corresponding to the observation image is generated by photoelectric conversion of the image sensor. The image signal generated by the imaging unit 5009 is provided to the communication unit 5013.

As the image sensor which is included by the imaging unit 5009, an image sensor, for example, of the complementary metal oxide semiconductor (CMOS) type, which has a Bayer array and is capable of imaging of an image in color is used. It is to be noted that, as the image sensor, an image sensor may be used which is ready, for example, for imaging of an image of a high resolution equal to or not less than 4K. If an image of a surgical location is obtained in a high resolution, then the surgeon 5067 can comprehend a state of the surgical location in enhanced details and can proceed with the surgery more smoothly.

Further, the image sensor which is included by the imaging unit 5009 includes such that it has a pair of image sensors for acquiring image signals for the right eye and the left eye compatible with 3D display. Where 3D display is applied, the surgeon 5067 can comprehend the depth of a living body tissue in the surgical location more accurately. It is to be noted that, if the imaging unit 5009 is configured as that of the multi-plate type, then a plurality of systems of lens units 5007 are provided corresponding to the individual image sensors of the imaging unit 5009.

The imaging unit 5009 may not necessarily be provided on the camera head 5005. For example, the imaging unit 5009 may be provided just behind the objective lens in the inside of the lens barrel 5003.

The driving unit 5011 includes an actuator and moves the zoom lens and the focusing lens of the lens unit 5007 by a predetermined distance along the optical axis under the control of the camera head controller 5015. Consequently, the magnification and the focal point of a captured image by the imaging unit 5009 can be adjusted suitably.

The communication unit 5013 includes a communication apparatus for transmitting and receiving various kinds of information to and from the CCU 5039. The communication unit 5013 transmits an image signal acquired from the imaging unit 5009 as RAW data to the CCU 5039 through the transmission cable 5065. Thereupon, in order to display a captured image of a surgical location in low latency, preferably the image signal is transmitted by optical communication. This is because, upon surgery, the surgeon 5067 performs surgery while observing the state of an affected area through a captured image, it is demanded for a moving image of the surgical location to be displayed on the real time basis as far as possible in order to achieve surgery with a higher degree of safety and certainty. Where optical communication is applied, a photoelectric conversion module for converting an electric signal into an optical signal is provided in the communication unit 5013. After the image signal is converted into an optical signal by the photoelectric conversion module, it is transmitted to the CCU 5039 through the transmission cable 5065.

Further, the communication unit 5013 receives a control signal for controlling driving of the camera head 5005 from the CCU 5039. The control signal includes information relating to image capturing conditions such as, for example, information that a frame rate of a captured image is designated, information that an exposure value upon image capturing is designated and/or information that a magnification and a focal point of a captured image are designated. The communication unit 5013 provides the received control signal to the camera head controller 5015. It is to be noted that also the control signal from the CCU 5039 may be transmitted by optical communication. In this case, a photoelectric conversion module for converting an optical signal into an electric signal is provided in the communication unit 5013. After the control signal is converted into an electric signal by the photoelectric conversion module, it is provided to the camera head controller 5015.

It is to be noted that the image capturing conditions such as the frame rate, exposure value, magnification or focal point are set automatically by the controller 5063 of the CCU 5039 on the basis of an acquired image signal. In other words, an auto exposure (AE) function, an auto focus (AF) function and an auto white balance (AWB) function are incorporated in the endoscope 5001.

The camera head controller 5015 controls driving of the camera head 5005 on the basis of a control signal from the CCU 5039 received through the communication unit 5013. For example, the camera head controller 5015 controls driving of the image sensor of the imaging unit 5009 on the basis of information that a frame rate of a captured image is designated and/or information that an exposure value upon image capturing is designated. Further, for example, the camera head controller 5015 controls the driving unit 5011 to suitably move the zoom lens and the focusing lens of the lens unit 5007 on the basis of information that a magnification and a focal point of a captured image are designated. The camera head controller 5015 may further include a function for storing information for identifying the lens barrel 5003 and/or the camera head 5005.

It is to be noted that, by disposing the components such as the lens unit 5007 and the imaging unit 5009 in a sealed structure having high airtightness and waterproof, the camera head 5005 can be provided with resistance to an autoclave sterilization process.

Now, a functional configuration of the CCU 5039 is described. The communication unit 5059 includes a communication apparatus for transmitting and receiving various kinds of information to and from the camera head 5005. The communication unit 5059 receives an image signal transmitted thereto from the camera head 5005 through the transmission cable 5065. Thereupon, the image signal may be transmitted preferably by optical communication as described above. In this case, for the compatibility with optical communication, the communication unit 5059 includes a photoelectric conversion module for converting an optical signal into an electric signal. The communication unit 5059 provides the image signal after conversion into an electric signal to the image processor 5061.

Further, the communication unit 5059 transmits, to the camera head 5005, a control signal for controlling driving of the camera head 5005. The control signal may also be transmitted by optical communication.

The image processor 5061 performs various image processes for an image signal in the form of RAW data transmitted thereto from the camera head 5005. The image processes include various known signal processes such as, for example, a development process, an image quality improving process (such as a bandwidth enhancement process, a super-resolution process, a noise reduction (NR) process and/or an image stabilization process) and/or an enlargement process (electronic zooming process). Further, the image processor 5061 performs a detection process for an image signal in order to perform AE, AF and AWB.

The image processor 5061 includes a processor such as a CPU or a GPU, and when the processor operates in accordance with a predetermined program, the image processes and the detection process described above can be performed. It is to be noted that, where the image processor 5061 includes a plurality of GPUs, the image processor 5061 suitably divides information relating to an image signal such that image processes are performed in parallel by the plurality of GPUs.

The controller 5063 performs various kinds of control relating to capturing an image of a surgical location by the endoscope 5001 and display of the captured image. For example, the controller 5063 generates a control signal for controlling driving of the camera head 5005. Thereupon, if image capturing conditions are input by the user, then the controller 5063 generates a control signal on the basis of the input by the user. Alternatively, where the endoscope 5001 has an AE function, an AF function and an AWB function incorporated therein, the controller 5063 suitably calculates an optimum exposure value, focal distance and white balance in response to a result of a detection process by the image processor 5061 and generates a control signal.

Further, the controller 5063 controls the display apparatus 5041 to display an image of a surgical location on the basis of an image signal for which image processes have been performed by the image processor 5061. Thereupon, the controller 5063 recognizes various objects in the surgical location image using various image recognition technologies. For example, the controller 5063 can recognize a surgical tool such as forceps, a particular living body region, bleeding, mist when the energy device 5021 is used and so forth by detecting the shape, color and so forth of edges of the objects included in the surgical location image. The controller 5063 causes, when it controls the display apparatus 5041 to display an image of the surgical location, various kinds of surgery supporting information to be displayed in an overlapping manner with the image of the surgical location using a result of the recognition. Where surgery supporting information is displayed in an overlapping manner and presented to the surgeon 5067, the surgeon 5067 can proceed with the surgery more safety and certainty.

The transmission cable 5065 which connects the camera head 5005 and the CCU 5039 to each other is an electric signal cable ready for communication of an electric signal, an optical fiber ready for optical communication or a composite cable ready for both of electrical and optical communication.

Here, while, in the example illustrated, communication is performed by wired communication using the transmission cable 5065, the communication between the camera head 5005 and the CCU 5039 may be performed otherwise by wireless communication. Where the communication between the camera head 5005 and the CCU 5039 is performed by wireless communication, there is no necessity to lay the transmission cable 5065 in the operating room. Therefore, such a situation that movement of medical staff in the operating room is disturbed by the transmission cable 5065 can be eliminated.

An example of the endoscopic surgery system 5000 to which the technology according to the present disclosure can be applied has been described above. It is to be noted here that, the system to which the technology according to the present disclosure can be applied is not limited to the endoscopic system. For example, the technology according to the present disclosure may be applied to a flexible endoscopic surgery system for inspection or another system such as a microscopic system.

The technology according to the present disclosure is suitably applicable to the endoscope 5001 among the configurations described above. Specifically, it is possible to apply the technology according to the present disclosure in the case of displaying an image of a surgical location inside the body cavity of the patient 5071 captured by the endoscope 5001 on the display apparatus 5041. Applying the technology according to the present disclosure to the endoscope 5001 makes it possible to approximate the color shade of the specific-wavelength light captured image to the color shade of the white light captured image, displaying the specific-wavelength light captured image. This configuration makes it possible for the surgeon 5067 to view a high-precision specific-wavelength light captured image on the display apparatus 5041 in real-time during the operation, resulting in more safety operations.

### Second Application Example

Further, the technology according to the present disclosure is applicable to the microscopy system. The description is given below a microsurgery system, which is an example of such a microscopy system. The microscopic surgery system is used for so-called microsurgery that is performed while enlarging a minute region of a patient for observation.

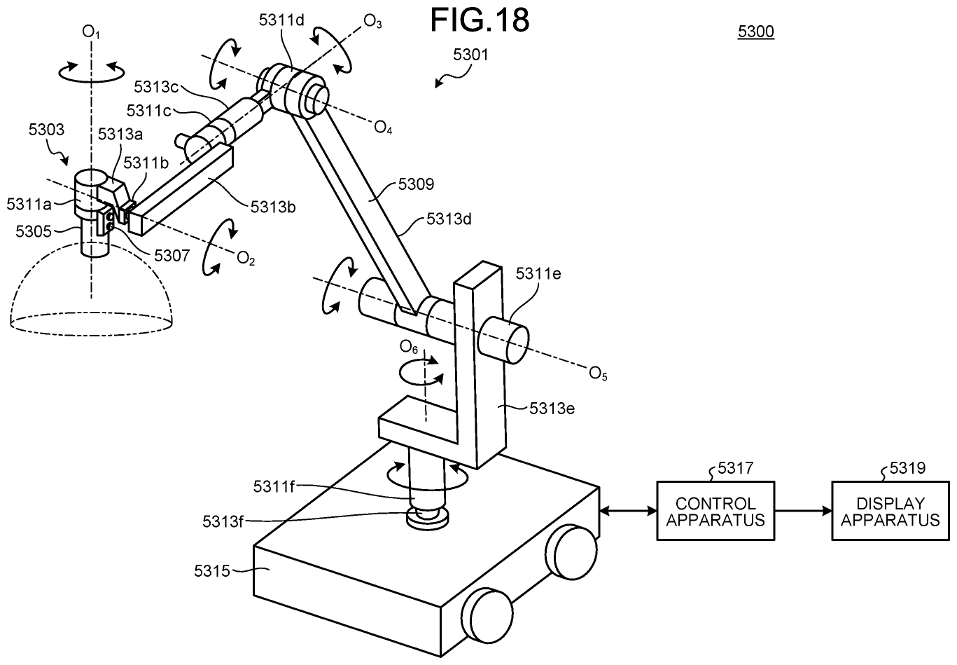

FIG. 18 is a view illustrating an example of a schematic configuration of a microscopic surgery system 5300 to which the technology according to the present disclosure can be applied. Referring to FIG. 18, the microscopic surgery system 5300 includes a microscope apparatus 5301, a control apparatus 5317 and a display apparatus 5319. It is to be noted that, in the description of the microscopic surgery system 5300, the term “user” signifies an arbitrary one of medical staff members such as a surgeon or an assistant who uses the microscopic surgery system 5300.

The Microscope apparatus 5301 has a microscope unit 5303 for enlarging an observation target (surgical location of a patient) for observation, an arm unit 5309 which supports the microscope unit 5303 at a distal end thereof, and a base unit 5315 which supports a proximal end of the arm unit 5309.

The microscope unit 5303 includes a cylindrical portion 5305 of a substantially cylindrical shape, an imaging unit (not illustrated) provided in the inside of the cylindrical portion 5305, and an operation unit 5307 provided in a partial region of an outer circumference of the cylindrical portion 5305. The microscope unit 5303 is a microscope unit of the electronic image capturing type (microscope unit of the video type) which captures a captured image electronically by the imaging unit.

A cover glass member for protecting the internal imaging unit is provided at an opening face of a lower end of the cylindrical portion 5305. Light from an observation target (hereinafter referred to also as observation light) passes through the cover glass member and enters the imaging unit in the inside of the cylindrical portion 5305. It is to be noted that a light source includes, for example, a light emitting diode (LED) or the like may be provided in the inside of the cylindrical portion 5305, and upon image capturing, light may be irradiated upon an observation target from the light source through the cover glass member.

The imaging unit includes an optical system which condenses observation light, and an image sensor which receives the observation light condensed by the optical system. The optical system includes a combination of a plurality of lenses including a zoom lens and a focusing lens. The optical system has optical properties adjusted such that the observation light is condensed to be formed image on a light receiving face of the image sensor. The image sensor receives and photoelectrically converts the observation light to generate a signal corresponding to the observation light, namely, an image signal corresponding to an observation image. As the image sensor, for example, an image sensor which has a Bayer array and is capable of imaging of an image in color is used. The image sensor may be any of various known image sensors such as a complementary metal oxide semiconductor (CMOS) image sensor or a charge coupled device (CCD) image sensor. The image signal generated by the image sensor is transmitted as RAW data to the control apparatus 5317. Here, the transmission of the image signal may be performed suitably by optical communication. This is because, since, at a surgery site, the surgeon performs surgery while observing the state of an affected area through a captured image, in order to achieve surgery with a higher degree of safety and certainty, it is demanded for a moving image of the surgical location to be displayed on the real time basis as far as possible. Where optical communication is used to transmit the image signal, the captured image can be displayed with low latency.

It is to be noted that the imaging unit may have a driving mechanism for moving the zoom lens and the focusing lens of the optical system thereof along the optical axis. Where the zoom lens and the focusing lens are moved suitably by the driving mechanism, the magnification of the captured image and the focal distance upon image capturing can be adjusted. Further, the imaging unit may incorporate therein various functions which may be provided generally in a microscope unit of the electronic image capturing type such as an auto exposure (AE) function or an auto focus (AF) function.

Further, the imaging unit may be configured as an imaging unit of the single-plate type which includes a single image sensor or may be configured as an imaging unit of the multi-plate type which includes a plurality of image sensors. Where the imaging unit is configured as that of the multi-plate type, for example, image signals corresponding to red, green, and blue colors may be generated by the image sensors and may be synthesized to obtain a color image. Alternatively, the imaging unit may be configured such that it has a pair of image sensors for acquiring image signals for the right eye and the left eye compatible with a stereoscopic vision (three dimensional (3D) display). Where 3D display is applied, the surgeon can comprehend the depth of a living body tissue in the surgical location with a higher degree of accuracy. It is to be noted that, if the imaging unit is configured as that of the multi-plate type, then a plurality of optical systems is provided corresponding to the individual image sensors.

The operation unit 5307 is input means that includes, for example, a cross lever, a switch or the like and accepts an operation input of the user. For example, the user can input an instruction to change the magnification of the observation image and the focal distance to the observation target through the operation unit 5307. The magnification and the focal distance can be adjusted by the driving mechanism of the imaging unit suitably moving the zoom lens and the focusing lens in accordance with the instruction. Further, for example, the user can input an instruction to switch the operation mode of the arm unit 5309 (an all-free mode and a fixed mode hereinafter described) through the operation unit 5307. It is to be noted that when the user intends to move the microscope unit 5303, it is supposed that the user moves the microscope unit 5303 in a state in which the user grasps the microscope unit 5303 holding the cylindrical portion 5305. Accordingly, the operation unit 5307 is preferably provided at a position at which it can be operated readily by the fingers of the user with the cylindrical portion 5305 held such that the operation unit 5307 can be operated even while the user is moving the cylindrical portion 5305.

The arm unit 5309 is configured such that a plurality of links (first link 5313a to sixth link 5313f) are connected for rotation relative to each other by a plurality of joint portions (first joint portion 5311a to sixth joint portion 5311f),

The first joint portion 5311a has a substantially columnar shape and supports, at a distal end (lower end) thereof, an upper end of the cylindrical portion 5305 of the microscope unit 5303 for rotation around an axis of rotation (first axis O1) parallel to the center axis of the cylindrical portion 5305. Here, the first joint portion 5311a may be configured such that the first axis O1 thereof is in alignment with the optical axis of the imaging unit of the microscope unit 5303. By the configuration, if the microscope unit 5303 is rotated around the first axis O1, then the field of view can be changed so as to rotate the captured image.

The first link 5313a fixedly supports, at a distal end thereof, the first joint portion 5311a. Specifically, the first link 5313a is a bar-like member having a substantially L shape and is connected to the first joint portion 5311a such that one side at the distal end side thereof extends in a direction orthogonal to the first axis O1 and an end portion of the one side abuts with an upper end portion of an outer periphery of the first joint portion 5311a. The second joint portion 5311b is connected to an end portion of the other side on the proximal end side of the substantially L shape of the first link 5313a.

The second joint portion 5311b has a substantially columnar shape and supports, at a distal end thereof, a proximal end of the first link 5313a for rotation around an axis of rotation (second axis O2) orthogonal to the first axis O1. The second link 5313b is fixedly connected at a distal end thereof to a proximal end of the second joint portion 5311b.

The second link 5313b is a bar-like member having a substantially L shape, and one side of a distal end side of the second link 5313b extends in a direction orthogonal to the second axis 02 and an end portion of the one side is fixedly connected to a proximal end of the second joint portion 5311b. The third joint portion 5311c is connected to the other side at the proximal end side of the substantially L shape of the second link 5313b.

The third joint portion 5311c has a substantially columnar shape and supports, at a distal end thereof, a proximal end of the second link 5313b for rotation around an axis of rotation (third axis O3) orthogonal to the first axis O1 and the second axis O2. The third link 5313c is fixedly connected at a distal end thereof to a proximal end of the third joint portion 5311c. By rotating the components at the distal end side including the microscope unit 5303 around the second axis O2 and the third axis O3, the microscope unit 5303 can be moved such that the position of the microscope unit 5303 is changed within a horizontal plane. In other words, by controlling the rotation around the second axis O2 and the third axis O3, the field of view of the captured image can be moved within a plane.

The third link 5313c is configured such that the distal end side thereof has a substantially columnar shape, and a proximal end of the third joint portion 5311c is fixedly connected to the distal end of the columnar shape such that both of them have a substantially same center axis. The proximal end side of the third link 5313c has a prismatic shape, and the fourth joint portion 5311d is connected to an end portion of the third link 5313c.

The fourth joint portion 5311d has a substantially columnar shape and supports, at a distal end thereof, a proximal end of the third link 5313c for rotation around an axis of rotation (fourth axis O4) orthogonal to the third axis O3. The fourth link 5313d is fixedly connected at a distal end thereof to a proximal end of the fourth joint portion 5311d.

The fourth link 5313d is a bar-like member extending substantially linearly and is fixedly connected to the fourth joint portion 5311d such that it extends orthogonally to the fourth axis O4 and abuts at an end portion of the distal end thereof with a side face of the substantially columnar shape of the fourth joint portion 5311d. The fifth joint portion 5311e is connected to a proximal end of the fourth link 5313d.

The fifth joint portion 5311e has a substantially columnar shape and supports, at a distal end side thereof, a proximal end of the fourth link 5313d for rotation around an axis of rotation (fifth axis O5) parallel to the fourth axis O4. The fifth link 5313e is fixedly connected at a distal end thereof to a proximal end of the fifth joint portion 5311e. The fourth axis O4 and the fifth axis O5 are axes of rotation around which the microscope unit 5303 can be moved in the upward and downward direction. By rotating the components at the distal end side including the microscope unit 5303 around the fourth axis O4 and the fifth axis O5, the height of the microscope unit 5303, namely, the distance between the microscope unit 5303 and an observation target, can be adjusted.

The fifth link 5313e includes a combination of a first member having a substantially L shape one side of which extends in the vertical direction and the other side of which extends in the horizontal direction, and a bar-like second member extending vertically downwardly from the portion of the first member which extends in the horizontal direction. The fifth joint portion 5311e is fixedly connected at a proximal end thereof to a neighboring upper end of a part of the first member of the fifth link 5313e extending in the vertical direction. The sixth joint portion 5311f is connected to a proximal end (lower end) of the second member of the fifth link 5313e.

The sixth joint portion 5311f has a substantially columnar shape and supports, at a distal end side thereof, a proximal end of the fifth link 5313e for rotation around an axis of rotation (sixth axis O6) parallel to the vertical direction. The sixth link 5313f is fixedly connected at a distal end thereof to a proximal end of the sixth joint portion 5311f.

The sixth link 5313f is a bar-like member extending in the vertical direction and is fixedly connected at a proximal end thereof to an upper face of the base unit 5315.

The first joint portion 5311a to sixth joint portion 5311f have rotatable ranges suitably set such that the microscope unit 5303 can make a desired movement. Consequently, in the arm unit 5309 having the configuration described above, a movement of totaling six degrees of freedom including three degrees of freedom for translation and three degrees of freedom for rotation can be implemented with regard to a movement of the microscope unit 5303. By configuring the arm unit 5309 such that six degrees of freedom are implemented for movements of the microscope unit 5303 in this manner, the position and the posture of the microscope unit 5303 can be controlled freely within the movable range of the arm unit 5309. Accordingly, it is possible to observe a surgical location from every angle, and surgery can be executed more smoothly.

It is to be noted that the configuration of the arm unit 5309 as illustrated is an example at all, and the number and shape (length) of the links and the number, location, direction of the axis of rotation and so forth of the joint portions included in the arm unit 5309 may be designed suitably such that desired degrees of freedom can be implemented. For example, in order to freely move the microscope unit 5303, preferably the arm unit 5309 is configured so as to have six degrees of freedom as described above. However, the arm unit 5309 may also be configured so as to have much greater degree of freedom (namely, redundant degree of freedom). Where a redundant degree of freedom exists, in the arm unit 5309, it is possible to change the posture of the arm unit 5309 in a state in which the position and the posture of the microscope unit 5303 are fixed. Accordingly, control can be implemented which is higher in convenience to the surgeon such as to control the posture of the arm unit 5309 such that, for example, the arm unit 5309 does not interfere with the field of view of the surgeon who watches the display apparatus 5319.

Here, an actuator in which a driving mechanism such as a motor, an encoder which detects an angle of rotation at each joint portion and so forth are incorporated may be provided for each of the first joint portion 5311a to sixth joint portion 5311f. By suitably controlling driving of the actuators provided in the first joint portion 5311a to sixth joint portion 5311f by the control apparatus 5317, the posture of the arm unit 5309, namely, the position and the posture of the microscope unit 5303, can be controlled. Specifically, the control apparatus 5317 can comprehend the posture of the arm unit 5309 at present and the position and the posture of the microscope unit 5303 at present on the basis of information regarding the angle of rotation of the joint portions detected by the encoders. The control apparatus 5317 uses the comprehended information to calculate a control value (for example, an angle of rotation or torque to be generated) for each joint portion with which a movement of the microscope unit 5303 in accordance with an operation input from the user is implemented. Accordingly, the control apparatus 5317 drives the driving mechanism of each joint portion in accordance with the control value. It is to be noted that, in this case, the control method of the arm unit 5309 by the control apparatus 5317 is not limited, and various known control methods such as force control or position control may be applied.

For example, when the surgeon performs operation inputting suitably through an inputting apparatus not illustrated, driving of the arm unit 5309 may be controlled suitably in response to the operation input by the control apparatus 5317 to control the position and the posture of the microscope unit 5303. By this control, it is possible to support, after the microscope unit 5303 is moved from an arbitrary position to a different arbitrary position, the microscope unit 5303 fixedly at the position after the movement. It is to be noted that, as the inputting apparatus, taking the convenience to the surgeon into consideration, an inputting apparatus which can be operated by the surgeon even if the surgeon has a surgical tool in its hand, such as, for example, a foot switch is preferably applied. Further, operation inputting may be performed in a contactless fashion on the basis of gesture detection or line-of-sight detection in which a wearable device or a camera which is provided in the operating room is used. This makes it possible even for a user who belongs to a clean area to operate an apparatus belonging to an unclean area with a high degree of freedom. In addition, the arm unit 5309 may be operated in a master-slave fashion. In this case, the arm unit 5309 may be remotely controlled by the user through an inputting apparatus which is placed at a place remote from the operating room.

Further, where force control is applied, the control apparatus 5317 may perform power-assisted control to drive the actuators of the first joint portion 5311a to sixth joint portion 5311f such that the arm unit 5309 may receive external force by the user and move smoothly following the external force. This makes it possible to move, when the user holds and directly moves the position of the microscope unit 5303, the microscope unit 5303 with comparatively weak force. Accordingly, it becomes possible for the user to move the microscope unit 5303 more intuitively by a simpler and easier operation, and the convenience to the user can be improved.

Further, driving of the arm unit 5309 may be controlled such that the arm unit 5309 performs a pivot movement. The pivot movement here is a motion for moving the microscope unit 5303 such that the direction of the optical axis of the microscope unit 5303 is kept toward a predetermined point (hereinafter referred to as pivot point) in a space. Since the pivot movement makes it possible to observe the same observation position from various directions, more detailed observation of an affected area becomes possible. It is to be noted that, where the microscope unit 5303 is configured such that the focal distance thereof is cannot be adjusted, preferably the pivot movement is performed in a state in which the distance between the microscope unit 5303 and the pivot point is fixed. In this case, it is sufficient if the distance between the microscope unit 5303 and the pivot point is adjusted to a fixed focal distance of the microscope unit 5303 in advance. By the configuration just described, the microscope unit 5303 comes to move on a hemispherical plane (schematically illustrated in FIG. 18) having a radius corresponding to the focal distance centered at the pivot point, and even if the observation direction is changed, a clear captured image can be obtained. On the other hand, where the microscope unit 5303 is configured such that the focal distance thereof is adjustable, the pivot movement may be performed in a state in which the distance between the microscope unit 5303 and the pivot point is variable. In this case, for example, the control apparatus 5317 may calculate the distance between the microscope unit 5303 and the pivot point on the basis of information regarding the angles of rotation of the joint portions detected by the encoders and automatically adjust the focal distance of the microscope unit 5303 on the basis of a result of the calculation. Alternatively, where the microscope unit 5303 includes an AF function, adjustment of the focal distance may be performed automatically by the AF function every time the changing in distance caused by the pivot movement between the microscope unit 5303 and the pivot point.

Further, each of the first joint portion 5311a to sixth joint portion 5311f may be provided with a brake for constraining the rotation of the first joint portion 5311a to sixth joint portion 5311f. Operation of the brake may be controlled by the control apparatus 5317. For example, if it is intended to fix the position and the posture of the microscope unit 5303, then the control apparatus 5317 renders the brakes of the joint portions operative. Consequently, even if the actuators are not driven, the posture of the arm unit 5309, namely, the position and posture of the microscope unit 5303, can be fixed, and therefore, the power consumption can be reduced. When it is intended to move the position and the posture of the microscope unit 5303, it is sufficient if the control apparatus 5317 releases the brakes of the joint portions and drives the actuators in accordance with a predetermined control method.

Such operation of the brakes may be performed in response to an operation input by the user through the operation unit 5307 described hereinabove. When the user intends to move the position and the posture of the microscope unit 5303, the user would operate the operation unit 5307 to release the brakes of the joint portions. Consequently, the operation mode of the arm unit 5309 changes to a mode in which rotation of the joint portions can be performed freely (all-free mode). On the other hand, if the user intends to fix the position and the posture of the microscope unit 5303, then the user would operate the operation unit 5307 to render the brakes of the joint portions operative. Consequently, the operation mode of the arm unit 5309 changes to a mode in which rotation of the joint portions is constrained (fixed mode).

The control apparatus 5317 integrally controls operation of the microscopic surgery system 5300 by controlling operation of the microscope apparatus 5301 and the display apparatus 5319. For example, the control apparatus 5317 renders the actuators of the first joint portion 5311a to sixth joint portion 5311f operative in accordance with a predetermined control method to control driving of the arm unit 5309. Further, for example, the control apparatus 5317 controls operation of the brakes of the first joint portion 5311a to sixth joint portion 5311f to change the operation mode of the arm unit 5309. Further, for example, the control apparatus 5317 performs various signal processes for an image signal acquired by the imaging unit of the microscope unit 5303 of the microscope apparatus 5301 to generate image data for display and controls the display apparatus 5319 to display the generated image data. As the signal processes, various known signal processes such as, for example, a development process (demosaic process), an image quality improving process (a bandwidth enhancement process, a super-resolution process, a noise reduction (NR) process and/or an image stabilization process) and/or an enlargement process (namely, an electronic zooming process) may be performed.

It is to be noted that communication between the control apparatus 5317 and the microscope unit 5303 and communication between the control apparatus 5317 and the first joint portion 5311a to sixth joint portion 5311f may be wired communication or wireless communication. Where wired communication is applied, communication by an electric signal may be performed or optical communication may be performed. In this case, a cable for transmission used for wired communication may be configured as an electric signal cable, an optical fiber or a composite cable of them in response to an applied communication method. On the other hand, where wireless communication is applied, since there is no necessity to lay a transmission cable in the operating room, such a situation that movement of medical staff in the operating room is disturbed by the transmission cable can be eliminated.

The control apparatus 5317 may be a processor such as a central processing unit (CPU) or a graphics processing unit (GPU), or a microcomputer or a control board in which a processor and a storage element such as a memory are incorporated. The various functions described hereinabove can be implemented by the processor of the control apparatus 5317 operating in accordance with a predetermined program. It is to be noted that, in the example illustrated, the control apparatus 5317 is provided as an apparatus separate from the microscope apparatus 5301. However, the control apparatus 5317 may be installed in the inside of the base unit 5315 of the microscope apparatus 5301 and configured integrally with the microscope apparatus 5301. The control apparatus 5317 may also include a plurality of apparatus. For example, microcomputers, control boards or the like may be disposed in the microscope unit 5303 and the first joint portion 5311a to sixth joint portion 5311f of the arm unit 5309 and connected for communication with each other to implement functions similar to those of the control apparatus 5317.

The display apparatus 5319 is provided in the operating room and displays an image corresponding to image data generated by the control apparatus 5317 under the control of the control apparatus 5317. In other words, an image of a surgical location imaged by the microscope unit 5303 is displayed on the display apparatus 5319. The display apparatus 5319 may display, in place of or in addition to an image of a surgical location, various kinds of information relating to the surgery such as physical information of a patient or information regarding a surgical procedure of the surgery. In this case, the display of the display apparatus 5319 may be switched suitably in response to an operation by the user. Alternatively, a plurality of such display apparatus 5319 may also be provided such that an image of a surgical location or various kinds of information relating to the surgery may individually be displayed on the plurality of display apparatus 5319. It is to be noted that, as the display apparatus 5319, various known display apparatus such as a liquid crystal display apparatus or an electro luminescence (EL) display apparatus may be applied.

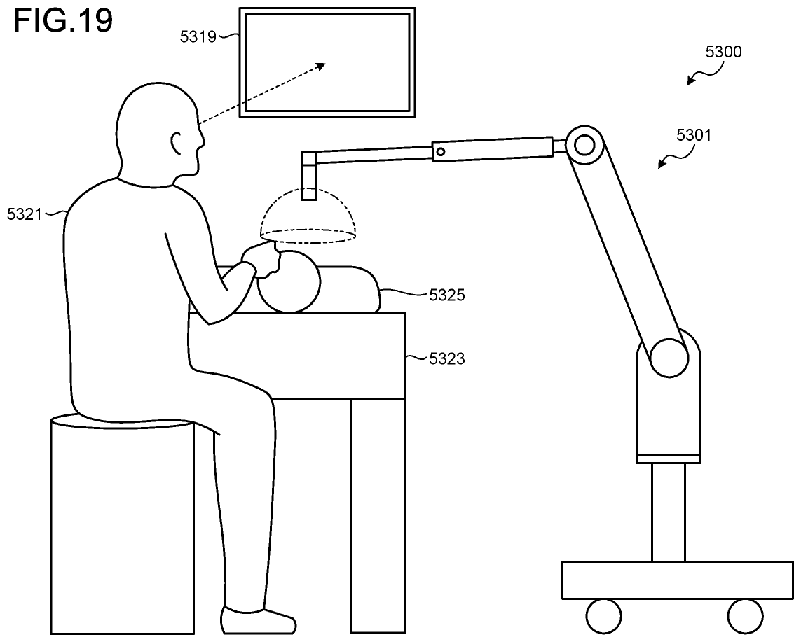

FIG. 19 is a view illustrating a state of surgery in which the microscopic surgery system 5300 illustrated in FIG. 18 is used. FIG. 19 schematically illustrates a state in which a surgeon 5321 uses the microscopic surgery system 5300 to perform surgery for a patient 5325 on a patient bed 5323. It is to be noted that, in FIG. 19, for simplified illustration, the control apparatus 5317 from among the components of the microscopic surgery system 5300 is omitted and the microscope apparatus 5301 is illustrated in a simplified from.

As illustrated in FIG. 2C, upon surgery, using the microscopic surgery system 5300, an image of a surgical location imaged by the microscope apparatus 5301 is displayed in an enlarged scale on the display apparatus 5319 installed on a wall face of the operating room. The display apparatus 5319 is installed at a position opposing to the surgeon 5321, and the surgeon 5321 would perform various treatments for the surgical location such as, for example, resection of the affected area while observing a state of the surgical location from a video displayed on the display apparatus 5319.

An example of the microscopic surgery system 5300 to which the technology according to the present disclosure can be applied has been described. It is to be noted here that, while the microscopic surgery system 5300 is described as an example, the system to which the technology according to the present disclosure can be applied is not limited to this example. For example, the microscope apparatus 5301 may also function as a supporting arm apparatus which supports, at a distal end thereof, a different observation apparatus or some other surgical tool in place of the microscope unit 5303. As the other observation apparatus, for example, an endoscope may be applied. Further, as the different surgical tool, forceps, tweezers, a pneumoperitoneum tube for pneumoperitoneum or an energy device for performing incision of a tissue or sealing of a blood vessel by cautery and so forth can be applied. By supporting any of such an observation apparatus and surgical tools as just described by the supporting arm apparatus, the position of them can be fixed with a high degree of stability in comparison with that in an alternative case in which they are supported by hands of medical staff. Accordingly, the burden on the medical staff can be reduced. The technology according to the present disclosure may be applied to a supporting arm apparatus which supports such a component as described above other than the microscope unit.

The technology according to the present disclosure is suitably applicable to the control apparatus 5317 among the configurations described above. Specifically, it is possible to apply the technology according to the present disclosure in the case of displaying an image indicating the surgical location of the patient 5325 that is captured by the imaging unit of the microscope unit 5303 on the display apparatus 5319. Applying the technology according to the present disclosure to the control apparatus 5317 makes it possible to approximate the color shade of the specific-wavelength light captured image to the color shade of the white light captured image, displaying the specific-wavelength light captured image. This configuration makes it possible for the surgeon 5321 to view a high-precision specific-wavelength light captured image on the display apparatus 5319 in real-time during the operation, resulting in more safety operations.

Note that the present technology may include the following configuration.

(1)

A medical system comprising:

a light source configured to irradiate an imaging target with light having different wavelength zones in a first observation mode and a second observation mode, the imaging target being a part of a living body being operated;

an imaging apparatus configured to capture reflected light from the imaging target irradiated with the light and output a captured image;

a storage controller configured to perform control for causing a storage section to store a first captured image upon the first observation mode as a reference image;

a generator configured to compare a second captured image upon the second observation mode with the reference image to generate a parameter used for approximating a color shade of the second captured image to a color shade of the reference image;

a color conversion processor configured to perform color conversion processing on the second captured image on a basis of the parameter to output a color-converted image; and

a display controller configured to perform control for causing a display section to display the color-converted image.

(2)

The medical system according to (1), wherein the storage controller performs the control for causing the storage section to store the first captured image upon the first observation mode as the reference image in a case where an area of an object other than the living body in an image is equal to or less than a predetermined proportion.

(3)

The medical system according to (1), wherein the storage controller performs the control for causing the storage section to store the first captured image upon the first observation mode as the reference image in a case where definition of an image is equal to or higher than a predetermined threshold.

(4)

The medical system according to (1), wherein the storage controller performs the control for causing the storage section to store the first captured image upon the first observation mode as the reference image in a case where a target part subjected to a surgical operation satisfies a predetermined condition in size and position.

(5)

The medical system according to (1), wherein the storage controller performs the control for causing the storage section to store the first captured image upon the first observation mode as the reference image at a timing specified by a user.

(6)

The medical system according to (1), wherein the storage controller performs control for causing the storage section to store a plurality of the first captured images upon the first observation mode as the reference images.

(7)

The medical system according to (1), wherein the generator generates the parameter in units of a pixel, units of a predetermined region including multiple pixels, or units of a whole image.

(8)

The medical system according to (7), wherein the generator generates the parameter by performing motion estimation and motion compensation on a subject to align positions of the subject in a case of generating the parameter in units of the pixel or units of the predetermined region.

(9)

The medical system according to (7), wherein the generator generates the parameter in such a way as to prevent discontinuity in a color shade at a boundary between pixels or between predetermined regions from occurring in a case of generating the parameter in units of the pixel or units of the predetermined region.

(10)

The medical system according to (1), wherein the generator identifies an internal organ in the second captured image to generate the parameter for each internal organ.

(11)

The medical system according to (1), wherein the generator generates the parameter as a parameter in a matrix format used to minimize a difference in color shade between an image subjected to color conversion and the reference image, the image subjected to color conversion being obtained upon subjecting the second captured image to color conversion.

(12)

The medical system according to (1), wherein the generator generates the parameter as a parameter in a lookup table format used to minimize a difference in color shade between an image subjected to color conversion and the reference image, the image subjected to color conversion being obtained upon subjecting the second captured image to color conversion.

(13)

The medical system according to (1), wherein the generator generates the parameter using machine learning.

(14)

The medical system according to (1), wherein the storage controller performs control for causing the storage section to store a color-converted image output by the color conversion processor, and

the generator generates the parameter further on a basis of the color-converted image stored in the storage section.

(15)

The medical system according to (1), wherein the generator generates the parameter in such a way as to approximate the color shade of the second captured image to the color shade of the reference image for a location of the living body specified by a user.

(16)

The medical system according to (1), wherein the first observation mode includes a white light observation mode, and the second observation mode includes a visible fluorescence observation mode.

(17)

The medical system according to (1), wherein the medical system includes a microscopy system or an endoscopy system.

(18)

An information processing apparatus operating in conjunction with a light source and an imaging apparatus, the apparatus comprising:

a storage controller configured to perform control for causing a storage section to store a first captured image upon a first observation mode as a reference image;

a generator configured to compare a second captured image upon a second observation mode with the reference image to generate a parameter used for approximating a color shade of the second captured image to a color shade of the reference image;

a color conversion processor configured to perform color conversion processing on the second captured image on a basis of the parameter to output a color-converted image; and

a display controller configured to perform control for causing a display section to display the color-converted image,

wherein the light source irradiates an imaging target with light having different wavelength zones in the first observation mode and the second observation mode, the imaging target being a part of a living body being operated, and

the imaging apparatus captures reflected light from the imaging target irradiated with the light and outputs a captured image.

(19)

An information processing method executed by an information processing apparatus operating in conjunction with a light source and an imaging apparatus, the method comprising:

a storage controlling step of performing control for causing a storage section to store a first captured image upon a first observation mode as a reference image;

a generating step of comparing a second captured image upon a second observation mode with the reference image to generate a parameter used for approximating a color shade of the second captured image to a color shade of the reference image;

a color conversion processing step of performing color conversion processing on the second captured image on a basis of the parameter to output a color-converted image; and

a display controlling step of performing control for causing a display section to display the color-converted image,

wherein the light source irradiates an imaging target with light having different wavelength zones in the first observation mode and the second observation mode, the imaging target being a part of a living body being operated, and

the imaging apparatus captures reflected light from the imaging target irradiated with the light and outputs a captured image.

Although the description above is give of the embodiments and modifications of the present disclosure, the technical scope of the present disclosure is not limited to the above-described embodiments and modifications as they are, and various modifications and variations can be made without departing from the spirit and scope of the present disclosure. In addition, components covering different embodiments and modifications can be combined as appropriate.

In one example, the objects to be combined as the first observation mode and the second observation mode are not limited to the white light observation mode and the visible fluorescence observation mode, but can include a combination of the white light observation mode and the specific-wavelength light observation mode other than the visible fluorescence observation mode or a combination of a predetermined reference observation mode and the observation mode targeted for color conversion.

Further, it is also possible to perform the color conversion independently in the case of using a plurality of specific-wavelength light observation modes.

Moreover, the effects in each of the embodiments and modifications described in the present specification are merely illustrative and are not restrictive, and other effects are achievable.

### REFERENCE SIGNS LIST

1 MEDICAL SYSTEM

2 LIGHT SOURCE

3 IMAGING APPARATUS

4 INFORMATION PROCESSING APPARATUS

5 DISPLAY APPARATUS

9 IMAGING TARGET

41 PROCESSOR

42 STORAGE SECTION

411 ACQUISITION SECTION

412 REFERENCE IMAGE STORAGE CONTROLLER

413 COLOR CONVERSION PARAMETER GENERATOR

414 COLOR CONVERSION PROCESSOR

415 DISPLAY CONTROLLER

416 COLOR-CONVERTED IMAGE STORAGE CONTROLLER

1. A medical system comprising:
a light source configured to irradiate an imaging target with light having different wavelength zones in a first observation mode and a second observation mode, the imaging target being a part of a living body being operated;
an imaging apparatus configured to capture reflected light from the imaging target irradiated with the light and output a captured image;
a storage controller configured to perform control for causing a storage section to store a first captured image upon the first observation mode as a reference image;
a generator configured to compare a second captured image upon the second observation mode with the reference image to generate a parameter used for approximating a color shade of the second captured image to a color shade of the reference image;
a color conversion processor configured to perform color conversion processing on the second captured image on a basis of the parameter to output a color-converted image; and
a display controller configured to perform control for causing a display section to display the color-converted image.

2. The medical system according to claim 1, wherein the storage controller performs the control for causing the storage section to store the first captured image upon the first observation mode as the reference image in a case where an area of an object other than the living body in an image is equal to or less than a predetermined proportion.

3. The medical system according to claim 1, wherein the storage controller performs the control for causing the storage section to store the first captured image upon the first observation mode as the reference image in a case where definition of an image is equal to or higher than a predetermined threshold.

4. The medical system according to claim 1, wherein the storage controller performs the control for causing the storage section to store the first captured image upon the first observation mode as the reference image in a case where a target part subjected to a surgical operation satisfies a predetermined condition in size and position.

5. The medical system according to claim 1, wherein the storage controller performs the control for causing the storage section to store the first captured image upon the first observation mode as the reference image at a timing specified by a user.

6. The medical system according to claim 1, wherein the storage controller performs control for causing the storage section to store a plurality of the first captured images upon the first observation mode as the reference images.

7. The medical system according to claim 1, wherein the generator generates the parameter in units of a pixel, units of a predetermined region including multiple pixels, or units of a whole image.

8. The medical system according to claim 7, wherein the generator generates the parameter by performing motion estimation and motion compensation on a subject to align positions of the subject in a case of generating the parameter in units of the pixel or units of the predetermined region.

9. The medical system according to claim 7, wherein the generator generates the parameter in such a way as to prevent discontinuity in a color shade at a boundary between pixels or between predetermined regions from occurring in a case of generating the parameter in units of the pixel or units of the predetermined region.

10. The medical system according to claim 1, wherein the generator identifies an internal organ in the second captured image to generate the parameter for each internal organ.

11. The medical system according to claim 1, wherein the generator generates the parameter as a parameter in a matrix format used to minimize a difference in color shade between an image subjected to color conversion and the reference image, the image subjected to color conversion being obtained upon subjecting the second captured image to color conversion.

12. The medical system according to claim 1, wherein the generator generates the parameter as a parameter in a lookup table format used to minimize a difference in color shade between an image subjected to color conversion and the reference image, the image subjected to color conversion being obtained upon subjecting the second captured image to color conversion.

13. The medical system according to claim 1, wherein the generator generates the parameter using machine learning.

14. The medical system according to claim 1, wherein the storage controller performs control for causing the storage section to store a color-converted image output by the color conversion processor, and
the generator generates the parameter further on a basis of the color-converted image stored in the storage section.

15. The medical system according to claim 1, wherein the generator generates the parameter in such a way as to approximate the color shade of the second captured image to the color shade of the reference image for a location of the living body specified by a user.

16. The medical system according to claim 1, wherein the first observation mode includes a white light observation mode, and the second observation mode includes a visible fluorescence observation mode.

17. The medical system according to claim 1, wherein the medical system includes a microscopy system or an endoscopy system.

18. An information processing apparatus operating in conjunction with a light source and an imaging apparatus, the apparatus comprising:
a storage controller configured to perform control for causing a storage section to store a first captured image upon a first observation mode as a reference image;
a generator configured to compare a second captured image upon a second observation mode with the reference image to generate a parameter used for approximating a color shade of the second captured image to a color shade of the reference image;
a color conversion processor configured to perform color conversion processing on the second captured image on a basis of the parameter to output a color-converted image; and
a display controller configured to perform control for causing a display section to display the color-converted image,
wherein the light source irradiates an imaging target with light having different wavelength zones in the first observation mode and the second observation mode, the imaging target being a part of a living body being operated, and
the imaging apparatus captures reflected light from the imaging target irradiated with the light and outputs a captured image.

19. An information processing method executed by an information processing apparatus operating in conjunction with a light source and an imaging apparatus, the method comprising:
a storage controlling step of performing control for causing a storage section to store a first captured image upon a first observation mode as a reference image;
a generating step of comparing a second captured image upon a second observation mode with the reference image to generate a parameter used for approximating a color shade of the second captured image to a color shade of the reference image;
a color conversion processing step of performing color conversion processing on the second captured image on a basis of the parameter to output a color-converted image; and
a display controlling step of performing control for causing a display section to display the color-converted image,
wherein the light source irradiates an imaging target with light having different wavelength zones in the first observation mode and the second observation mode, the imaging target being a part of a living body being operated, and
the imaging apparatus captures reflected light from the imaging target irradiated with the light and outputs a captured image.
